(19) United States
(12) Patent Application Publication
(10) Pub. No.: US 2004/0157314 A1
(43) Pub. Date: Aug. 12, 2004

(54) Inhibitors of Staphylococcus aureus primary sigma factor and uses thereof

(75) Inventors: Dominique Bergeron, Montreal (CA); Mohammed Dehbi, Laval (CA); Michael Dubow, Antony (FR); Philippe Gros, St-Lambert (CA); John McCarty, Lyndonville, VT (US); Jerry Pelletier, Baie-d'Urfe (CA)

Correspondence Address: SUGHRUE MION, PLLC, 2100 PENNSYLVANIA AVENUE, N.W., SUITE 800, WASHINGTON, DC, 20037, US

(73) Assignee: PHAGETECH INC.

(21) Appl. No.: 10/771,395

(22) Filed: Feb. 5, 2004

Related U.S. Application Data
(60) Provisional application No. 60/445,441, filed on Feb. 7, 2003.

(51) Int. Cl. C12N 1/20

(52) U.S. Cl. 435/252.3

(57) ABSTRACT

The present invention discloses inhibitors of the primary sigma factor in Staphylococcus aureus (σ^{SA}), an essential protein implicated in RNA synthesis. The invention also discloses methods for inhibiting bacterial growth by contacting a bacterium with an antibacterial compound that specifically binds to a bacteriophage polypeptide binding domain of the Staphylococcus aureus primary sigma factor. Examples of inhibitors include bacteriophage polypeptides inhibiting growth of S. aureus and bacteriophage polypeptides binding specifically to S. aureus primary sigma factor. In addition, the invention discloses screening methods for identifying compounds which inhibit biochemical and/or cellular activity of S. aureus primary sigma factor.

### RELATED APPLICATION

This application claims priority from U.S. Provisional Application 60/445,441, filed Feb. 7, 2003, the disclosure of which is incorporated by reference herein in its entirety.

### BACKGROUND OF THE INVENTION

a) Field of the Invention

The invention relates to antibacterial compounds and particularly to inhibitors of primary sigma factor in Staphylococcus aureus (σ^{SA}), an essential protein implicated in RNA synthesis.

In addition, the invention relates to screening assays to identify compounds which inhibit biochemical and cellular activity of S. aureus primary sigma factor (σ^{SA}).

b) Brief Description of the Prior Art

The Staphylococci make up a medically important genus of microbes known to cause several types of diseases in humans. Staphylococcus aureus is a Gram positive organism which can be found on the skin of healthy human hosts and it is responsible for a large number of bacteremias. Bacteria are now becoming increasingly resistant to common antibiotics and it is not uncommon to isolate S. aureus strains which are resistant to most, if not all, of the known antibiotics. Therefore, there is an unmet medical need and demand for new anti-microbial compounds, vaccines, drug screening methods, and diagnostic tools for this organism.

Factors governing bacterial cell-life regulation have become increasingly attractive targets for discovery of anti-infective drugs. Transcription is a process essential for cell life by which genes are transcribed to produce RNA. In prokaryotes, transcription is catalyzed by the DNA-dependent RNA polymerase enzyme, a multi-complex of polypeptides consisting of a core RNA polymerase (α2, β and β′ subunits) which interacts with one of the multiple species of sigma factor to form the holoenzyme or the transcription machinery. Given that the core enzyme by itself binds weakly and non specifically to DNA, the requirement of sigma factor is thus crucial for directing the core enzyme for specific promoter recognition and therefore for efficient initiation of transcription. Given its essentiality, sigma factor is a good target for novel antibacterial compounds. This is one main reason why methods and recombinant bacteria have been developed for screening antibacterial compounds targeting the sigma factor in E. coli (U.S. Pat. No. 6,613,531 to Wisconsin Alumni Research Found.) as well as in S. aureus (U.S. Pat. No. 6,451,582 to Anadys Pharmaceuticals).

Many sigma factors have been described in E. coli (at least seven) and B. subtilis (at least eighteen) (Morikawa et al., Genes to Cells (2003) #8: 699-712). In S. aureus, only three (3) sigma factors have been described so far: the primary sigma factor SA “σ^{SA}” (Morikawa et al., (2003)), the orthologue of E. coli sigma factor 70 (σ⁷⁰) (Rao et al., J Bacteriol. (1995) #177:2609-2614; Deora et Misra J Biol Chem. (1996) #271:21828-21834) and the alternative factor sigma B“σ^{B}” (U.S. Pat. No. 6,310,192; Wu et al., J Bacteriol. (1996) #178:6036-6042). Both factors have been shown to stimulate transcription by the core RNA polymerase enzyme from specific promoters (Rao et al., J Bacteriol. (1995) #177:2609-2614; Deora et Misra J Biol Chem. (1996) #271:21828-21834; Deora et al, J. Bacteriol. (1997) #179: 6355-6359). The primary S. aureus sigma factor (σ^{SA}) is encoded by the plaC gene (GenBank™ acc. No. M63177) and its amino acid sequence (GenBank™ acc. No. AAB59090) shares 79% identity with the vegetative sigma factor A (σ^{A}) of B. subtilis (GenBank™ acc. No. CAB14463) and 55% identity with the σ⁷⁰ of E. coli (GenBank™ acc. No. BAB37373).

A variety of antagonists of some sigma factors called “antisigma” have been described for E. coli, B. subtilis and to a lesser extend in S. aureus. However, in most of the cases, those antisigmas are directed against the alternative sigma factors (e.g. RsbW against Sigma B in S. aureus). So far, the only known anti-sigma factor against primary sigma factor is encoded by E. coli's bacteriophage T4 and this anti-sigma factor corresponds to the AsiA protein (GenBank™ acc. No. NP_{—}049866). AsiA has been suggested to be useful for the treatment of infective diseases (International PCT application WO 96/25170 to Research Found. of State Univ. NY) and for identifying ligands to E. coli RNA polymerase sigma 70 subunit (International PCT applications WO 99/64866 to Astra AB). Also in E. coli, International PCT application WO 99/43338 to Metastat Inc. discloses a 22 amino acids peptide corresponding to a fragment from the RNA polymerase β′ subunit, this peptide binding both E. coli σ³⁸ and σ⁷⁰ and inhibiting in vitro bacterial growth. However, prior to the present invention, no one has ever found an anti-sigma factor for S. aureus primary sigma factor (σ^{SA}), or any antibacterial compound capable of inhibiting σ^{SA}-dependent RNA polymerase activity.

In view of the above, there is a need for a first anti-primary sigma factor in S. aureus. There is also a need for antibacterial compounds or bacterial growth-inhibitory compounds (inhibitors) capable of inhibiting or blocking S. aureus primary sigma factor (σ^{SA}) biochemical and/or cellular functions.

There also remains a need to identify new antimicrobial compounds, screening assays and therapeutic methods targeting S. aureus primary sigma factor (σ^{SA}).

The present invention fulfills these needs and also other needs as it will be apparent to those skilled in the art upon reading the following specification.

### SUMMARY OF THE INVENTION

The present inventors have discovered inhibitors (anti-sigma factors) of the primary sigma factor in Staphylococcus aureus (USA; also called herein STAAU_R12). In one embodiment, the novel anti-primary sigma factors correspond to a 198 amino acids protein (herein called “G1ORF 67”, SEQ ID NO: 7) from S. aureus bacteriophage G1 and to a 149 amino acid fragment of that bacteriophage protein (SEQ ID NO: 8). The present inventors have clearly demonstrated, both in biochemical and in cellular assays, that: (i) G1ORF67 has a bacteriostatic effect on Staphylococcus aureus; (ii) G 1ORF67 and its fragment physically interact with σ^{SA}; (iii) σ^{SA}-dependent DNA binding activity is inhibited by G1ORF67; (iv) σ^{SA}-dependent transcriptional activity is inhibited by G1ORF67; and (v) it is the specific interaction between σ^{SA} and G1ORF67 which negatively modulate σ^{SA} function. Additionally, the inventors have identified a 195 amino acid protein from S. aureus bacteriophage Twort (herein called “TwortORF65”, SEQ ID NO: 10) which shares significant homology to G1ORF67. Therefore, TwortORF65 is likely another anti-primary sigma factor, potentially having binding and inhibitory activity similar to that of G1ORF67. The invention also encompasses anti-primary sigma factors included within the definition of a G1ORF67/TwortORF65-consensus sequence as set forth in SEQ ID NO: 12.

Therefore, according to a first aspect, the invention features an anti-sigma factor which inhibits or blocks the physiological function (i.e. the biochemical and/or cellular activity) of the S. aureus primary sigma factor (σ^{SA}), or the activity of a biologically active fragment or variant thereof.

According to another aspect, the invention concerns an antibacterial compound which inhibits, in S. aureus, σ^{SA}-dependent RNA polymerase activity.

According to a further aspect, the invention features an antibacterial compound which inhibits or blocks the DNA binding activity of the S. aureus primary sigma factor (σ^{SA}), or of a biologically active fragment or variant thereof.

Yet another aspect the invention features an antibacterial compound which inhibits interaction between: (i) S. aureus primary sigma factor (σ^{SA}) or of a biologically active fragment or variant thereof; and (ii) S. aureus Core-RNA polymerase.

Yet another aspect of the invention concerns an antibacterial compound having a bactericidal or bacteriostatic effect on Staphylococcus aureus, this antibacterial compound binding to a polypeptide comprising the amino acid sequence of SEQ ID NO: 2 (σ^{SA}).

In one embodiment, the anti-primary sigma factor and/or the antibacterial compound defined hereinbefore binds a bacteriophage binding domain of S. aureus primary sigma factor. Preferably, the anti-primary sigma factor and/or the antibacterial compound mimics the inhibitory activity and/or the bactericidal or bacteriostatic effect of G1ORF67.

The anti-primary sigma factor and/or the antibacterial compound defined hereinbefore may be a small molecule, a peptidomimetic compound, or a polypeptide. Suitable polypeptides include bacteriophage polypeptides such as SEQ ID NO: 7 (G1ORF67), SEQ ID NO: 8 (AA 50-198 of G1ORF67), and biologically active fragments and variants thereof binding to the polypeptide set forth in SEQ ID NO: 2 and/or inhibiting S. aureus growth. According to further aspect, the invention features an anti-infective composition comprising an antibacterial compound and/or an anti-primary sigma factor as defined hereinbefore, and a pharmaceutically acceptable carrier or diluent.

The invention also features methods for inhibiting bacterial growth, preferably S. aureus growth. In one embodiment, the method comprises contacting a bacterium with an antibacterial compound that specifically binds to a bacteriophage polypeptide binding domain of Staphylococcus aureus primary sigma factor polypeptide.

In another embodiment, the method for inhibiting bacterial growth comprises contacting the bacterium with an antibacterial compound, and/or an anti-primary sigma factor and/or an anti-infective composition as defined hereinbefore.

The invention also features an isolated or purified bacterial polypeptide fragment of the S. aureus primary sigma factor (σ^{SA}) set forth in SEQ ID NO: 2, this bacterial polypeptide fragment comprising a bacteriophage polypeptide binding domain. The bacterial polypeptide fragment may comprise as few as 5 amino acids (preferably from about 25 to about 245 amino acids) and up to 367 amino acids. Preferably, the bacteriophage polypeptide binding domain binds a polypeptide from bacteriophage G1, more preferably the bacteriophage polypeptide set forth in SEQ ID NO: 7 (G1ORF67) and the bacteriophage polypeptide consisting of amino acids 50-198 of G1ORF67 (SEQ ID NO: 8). In a preferred embodiment, the bacteriophage polypeptide binding domain comprises an amino acid sequence selected from the group consisting of amino acids 127-368 of ₀SA (SEQ ID NO: 3), amino acids 294-368 of σ^{SA} (SEQ ID NO: 4) and amino acids 294-360 of σ^{SA} (SEQ ID NO: 5).

The invention also concerns an isolated, purified or enriched nucleic acid molecule comprising nucleotides 1-597 of SEQ ID NO:6 encoding the G1ORF67 polypeptide. The invention further concerns an isolated, purified or enriched nucleic acid molecule comprising a polynucleotide fragment of at least 15, 25, 50, 75, 100, 150, 200, 300, 400, 500, 550 or more contiguous nucleotides of the bacteriophage nucleic acid sequence set forth in SEQ ID NO: 6 (G1ORF67). In addition, the invention concerns an isolated, purified or enriched nucleic acid molecule variant that has at least 50%, 60%, 70%, 75%, 80%, 85%, 90%, 95%, 95% and even 100% sequence identity with (1) the polynucleotide set forth in SEQ ID NO:6, (2) a fragment of the polynucleotide set forth in SEQ ID NO:6, or (3) a nucleic acid sequence encoding an amino acid sequence as set forth in SEQ ID NO: 7, SEQ ID NO: 8, or with the complement thereof.

The invention is further concerned with a substantially pure polynucleotide that hybridizes under stringent hybridization conditions with the complement of the polynucleotide set forth in SEQ ID NO:6, (2) the complement of a fragment of the polynucleotide set forth in SEQ ID NO:6, (3) the complement of a nucleic acid sequence encoding the amino acid sequence set forth in SEQ ID NO:7, or (4) the complement of a nucleic acid sequence encoding the amino acid sequence set forth in SEQ ID NO:8.

A related aspect of the invention concerns isolated or purified bacteriophage polypeptides. In one embodiment, the bacteriophage polypeptide of the invention binds the Staphylococcus aureus primary sigma factor polypeptide set forth in SEQ ID NO: 2. In another embodiment, the bacteriophage polypeptide comprises amino acids 1-198 of SEQ ID NO:7 (G1ORF67). In a further embodiment, the bacteriophage polypeptide comprises a polypeptide fragment of at least 10, 20, 30, 40, 50, 75, 100, 150 or more contiguous amino acids of the amino acid sequence set forth in SEQ ID NO: 7 (G1ORF67). In addition, the invention concerns a polypeptide variant that has at least 50%, 60%, 70%, 75%, 80%, 85%, 90%, 95%, 95% and even 100% sequence identity with (1) the amino acid sequence set forth in SEQ ID NO: 7, (2) the amino acid sequence set forth in SEQ ID NO: 8, or (3) a fragment of SEQ ID NO:7, wherein the variant binds the polypeptide set forth in SEQ ID NO: 2 and/or inhibiting growth of S. aureus. The invention is further concerned with a substantially pure polypeptide resulting from recombinant expression of a polynucleotide that hybridizes under stringent hybridization conditions with the complement of the polynucleotide set forth in SEQ ID NO:6, (2) the complement of a fragment of the polynucleotide set forth in SEQ ID NO:6, (3) the complement of a nucleic acid sequence encoding the amino acid sequence set forth in SEQ ID NO:7, or (4) the complement of a nucleic acid sequence encoding the amino acid sequence set forth in SEQ ID NO:8.

According to another aspect, the invention features screening methods. In one embodiment, the screening method comprises the steps of:

(a) contacting a Staphylococcus aureus primary sigma factor polypeptide comprising a bacteriophage binding domain with a test compound in the presence of a bacteriophage polypeptide that specifically binds to the bacteriophage binding domain; and

(b) determining whether the test compound inhibits binding of the bacteriophage polypeptide to the bacteriophage binding domain.

In another embodiment, the method comprises the steps of:

(a) contacting (i) a first polypeptide binding domain, (ii) a second polypeptide binding domain and (iii) at least one test compound, wherein the first and second polypeptide binding domains bind specifically with each other,

wherein the first polypeptide binding domain comprises an amino acid sequence selected from the group consisting of: the amino acid sequence set forth in SEQ ID NO: 3; the amino acid sequence set forth in SEQ ID NO: 4; and the amino acid sequence set forth in SEQ ID NO: 5;

wherein the second polypeptide binding domain comprises the amino acid sequence set forth in SEQ ID NO: 7 or SEQ ID NO: 8; and

(b) determining whether said at least one test compound inhibits binding between said first and second polypeptide binding domains.

In a related aspect, the invention features a method of making an antibacterial compound, the method comprising the steps of:

identifying a compound which interacts with a S. aureus primary sigma factor polypeptide by carrying out a screening method as defined previously; and

synthesizing or purifying the compound identified, preferably in an amount sufficient to provide a therapeutic or prophylactic effect when administered to an organism infected by S. aureus.

The invention further encompasses all aspects of the invention relating to G1ORF67, but instead of using G1ORF67, one use TwortORF65 (or a fragment or variant or homologue thereof) and/or use any polypeptide (or a fragment or variant or homologue thereof) comprising a G1ORF67/TwortORF65-consensus sequence as set forth in SEQ ID NO: 12.

One of the greatest advantages of the present invention is that it provides inhibitors of the activity or function of the primary sigma factor in Staphylococcus aureus (σ^{SA}) that could be useful as antibacterial compounds. The invention also provides screening assays for identifying further inhibitors of σ^{SA} and/or of RNA polymerase functions.

Other objects, advantages and features of the present invention will become more apparent upon reading of the following non-restrictive description of preferred embodiments with reference to the accompanying drawings which are exemplary and should not be interpreted as limiting the scope of the present invention.

### BRIEF DESCRIPTION OF THE DRAWINGS

FIG. 1 is a line graph illustrating the bacteriostatic effect of G1ORF67 in S. aureus.

FIG. 2 is a picture of an SDS-PAGE gel illustrating the S. aureus protein (PT46; STAAU_R12) interacting with G1ORF67. Lane 1: Molecular weight markers; Lanes 2: Lysate alone; Lanes 3: GST plus lysate; Lanes 4: G1ORF67 plus lysate; Lane 5: G1ORF67 minus lysate.

FIG. 3A is a schema illustrating deletions of S. aureus primary sigma factor (σ^{SA}) polypeptide (STAAU_R12) and the interaction of the deleted polypeptides with full length G1ORF67 as demonstrated in a yeast two-hybrid assay.

FIG. 3B is a schema illustrating deletions of G1ORF67 and the interaction of the deleted polypeptides with full length STAAU_R12 (σ^{SA}) as demonstrated in a yeast two-hybrid assay.

FIG. 4 is a picture of an autoradiogram showing the interaction STAAU_R12 (σ^{SA}) and G1ORF67 in a Far Western assay. Lane 1: 100 ng G1ORF67; Lane 2:250 ng G1ORF67; Lane 3: 500 ng G1ORF67; Lane 4: 1 μg G1ORF67; Lane 5: 2 μg G1ORF67; Lane 6: 2 μg 77ORF104.

FIG. 5A is a bar graph confirming the interaction between STAAU_R12 and G1ORF67 as measured by TR-FRET.

FIG. 5B is a line graph illustrating measured IC₅₀ of the interaction between STAAU_R12 and G1ORF67 as measured by TR-FRET. Results are shown as the mean of duplicates±S.D.

FIGS. 6A, 6B and 6C are pictures of polyacrylamide/urea gels showing results of in vitro transcription studies. FIG. 6A shows STAAU_R12-dependent in vitro transcription activity using an holoenzyme reconstituted from STAAU_R12 and E. coli core RNA polymerase. FIG. 6B shows specific inhibition by G1ORF67 of the STAAU_R12-dependent in vitro transcription. FIG. 6C shows that G1ORF67 has no inhibitory effect of in vitro transcriptional activity of E. coli holoenzyme (E. coli sigma factor and E. coli core RNA polymerase).

FIG. 7 is a schema illustrating a preferred embodiment of an optimized High-Throughput in vitro biochemical assay for screening compounds inhibiting RNA synthesis.

FIG. 8 is a bar graph showing results of a TCA precipitation assay in a 96-well format confirming that in vitro transcription of S. aureus RNA polymerase is STAAU_R12-dependent. Bar 1: E. coli core enzyme plus STAAU_R12; Bar 2: E. coli core enzyme plus STAAU_R12 and G1ORF67; Bar 3: E. coli core enzyme plus STAAU_R12 plus GST control; Bar 4: E. coli core enzyme plus STAAU_R12 and RNAse A.

FIG. 9 is a picture of an electrophoretic mobility shift assay showing STAAU_R12-dependent DNA binding activity of STAAU_R12 and inhibition by G1ORF67.

FIG. 10 is a bar graph confirming the interaction between STAAU_R12 and DNA as measured by TR-FRET.

FIG. 11 is a line graph illustrating the inhibition of transcription by G1ORF67 as measured by uridine uptake in S. aureus.

FIG. 12 is an alignment of G1ORF67 (SEQ ID NO: 7) and TwortORF65 (SEQ ID NO: 10) amino acid sequences.

### DETAILED DESCRIPTION OF THE INVENTION

A) Definitions

Throughout the text, the word “kilobase” is generally abbreviated as “kb”, the words “deoxyribonucleic acid” as “DNA”, the words “ribonucleic acid” as “RNA”, the words “complementary DNA” as “cDNA”, the words “polymerase chain reaction” as “PCR”, and the words “reverse transcription” as “RT”. Nucleotide sequences are written in the 5′ to 3′ orientation unless stated otherwise.

In order to provide an even clearer and more consistent understanding of the specification and the claims, including the scope given herein to such terms, the following definitions are provided:

Antibacterial agent or Antibacterial Compound: As used herein, the term “antibacterial agent” or “antibacterial compound” refers to an agent or compound that has a bactericidal or bacteriostatic effect on one or more bacterial strains, excluding naturally occurring or derived bacterial polypeptides. Preferably such an agent or compound is bactericidal or bacteriostatic on at least S. aureus. The antibacterial compound may be directly active on a S. aureus STAAU_R12 polypeptide, or it may be active on one or more constituents in a pathway that leads to reduced or decreased activity or function of a S. aureus STAAU_R12 polypeptide.

Anti-primary sigma factor: As used herein, this term refers to a repressor or antagonist of S. aureus primary sigma factor (σ^{SA}) activity, excluding naturally occurring or derived bacterial polypeptides. The term also includes those compounds that while not having a direct effect on the activity of USA, bind to σ^{SA} and thereby interfere with interactions between σ^{SA} and its binding partners.

Binding or binding interaction or interaction: As used herein, it refers to a physical association between two molecules involving contact between the two molecules (e.g. protein:protein; protein:polynucleotide; chemical compound:protein, etc). The term “specifically binding” or “specific interaction” in the context of the interaction of one or two polypeptides means that the polypeptide(s) have a measurable affinity for a binding partner (e.g. protein, polynucleotides; chemical compound, etc). This generally means that the polypeptide(s) physically interact via discrete regions or domains on the polypeptide(s), wherein the interaction is dependent upon the amino acid sequence(s) of the interacting domain(s). Binding specificity or affinity of two molecules can be normally be measured using methods and techniques well know in the art.

Biological activity or function: Refers to a detectable biochemical, cellular activity or physiological function attributable to a polypeptide. As used herein, it generally refers to Staphylococcus aureus primary sigma factor (or STAAU_R12) biological activity including but not limited to DNA binding activity, interaction with Core-RNA polymerase, σ^{SA}-mediated transcriptional activation and RNA polymerization. Inhibition or Decrease in activity refers to a reduced level of measurable activity of a polypeptide in a given assay with suitable controls. Activity is considered decreased according to the invention if it is at least 10% less, preferably 15% less, 20% less, 50% less, 75% less, 90% less or even 100% less (i.e., no activity) than the activity under control conditions.

Derived from: as used herein, it generally refers to a polypeptide which shares a substantial level of identity at the amino acids level (from 50 to 100%) with a “reference” or “original” polypeptide or to a portion thereof. This includes, among other things, fragments and variants obtained by addition, deletion, or substitution of one or more amino acids of the “reference” or “original” polypeptides.

Fragment: Refers to a portion of a molecule that is less than the entire or full-length molecule, where the molecule is a generally a biomolecule such as a protein, a polypeptide or a polynucleotide. A fragment refers to any portion of the molecule, of any size, including a single amino acid or nucleotide. A “biologically active fragment” refers to a fragment of a molecule having at least a portion of the original biological activity of the entire or full-length molecule, preferably substantially the same level or more preferably an improved activity, or having a decreased undesirable activity when compared to the full-length molecule.

Inhibit or inhibition or inhibitory or inhibitor: Refer to a function of reducing a biological activity or function. Such reduction in activity or function can, for example, be in connection with a cellular component (e.g., an enzyme), or in connection with a cellular process (e.g. transcription, binding) or in connection with an overall process of a cell (e.g. cell growth). In reference to cell growth, the inhibitory effects may be bactericidal (killing of bacterial cells) or bacteriostatic (i.e.—stopping or at least slowing bacterial cell growth). The latter slows or prevents cell growth such that fewer cells of the strain are produced relative to uninhibited cells over a given time period. From a molecular standpoint, such inhibition may equate with a reduction in the level of, or elimination of, the transcription and/or translation and/or stability and/or binding of a specific bacterial target(s), and/or reduction or elimination of activity of a particular target molecule. Activity, function or binding is considered inhibited or decreased according to the invention if it is at least 10% less, preferably 15% less, 20% less, 50% less, 75% less, 90% less or even 100% less (i.e., no activity) than the activity, function or binding under control conditions.

Isolated or Purified: Means altered “by the hand of man” from its natural state (i.e. if it occurs in nature, it has been changed or removed from its original environment) or it has been synthesized in a non-natural environment (e.g., artificially synthesized). These terms do not require absolute purity (such as a homogeneous preparation) but instead represents an indication that it is relatively more pure than in the natural environment. For example, a polynucleotide or a protein/peptide naturally present in a living organism is not “isolated”, but the same polynucleotide separated (about 90-95% pure at least) from the coexisting materials of its natural state, obtained by cloning, amplification and/or chemical synthesis is “isolated” or “purified” as these terms are employed herein. Moreover, a polynucleotide or a protein/peptide that is introduced into an organism by transformation, genetic manipulation or by any other recombinant method is “isolated” even if it is still present in said organism.

Mimetic: refers to a compound that can be natural, synthetic, or chimeric and is structurally and functionally related to a reference compound. For instance a “Peptidomimetic” is a non-peptide compound that mimics the activity-related aspects of the 3-dimensional structure of a peptide or polypeptide, such as a compound that mimics the structure of a peptide or active portion of a phage ORF-encoded polypeptide.

Nucleic acid or polynucleotide: Any DNA sequence, RNA sequence or molecule having two nucleotides or more, including nucleotide sequences encoding a complete gene. The term is intended to encompass all nucleic acids whether occurring naturally or non-naturally in a particular cell, tissue or organism. This includes DNA and fragments thereof, RNA and fragments thereof, cDNAs and fragments thereof, expressed sequence tags, artificial sequences including randomized artificial sequences, and hybrid molecules.

ORF65 or phage Twort ORF65 or TwortORF65: refers to a polypeptide encoded by SEQ ID NO: 9, or to a fragment or variant thereof encoding a polypeptide having a biological activity substantially similar to TwortORF65 set forth in SEQ ID NO: 10.

ORF67 or phage G1ORF67 or G1ORF67: refers to a polypeptide encoded by SEQ ID NO: 6, or to a fragment or variant thereof encoding a polypeptide having a biological activity substantially similar to G1ORF67 set forth in SEQ ID NO: 7.

Peptide or Polypeptide: means any chain of more than two amino acids joined to each other by peptide bonds or modified peptide bonds, regardless of post-translational modification such as glycosylation or phosphorylation. Polypeptides may contain natural or synthetic amino acids other than the 20 gene-encoded amino acids and they may be branched or cyclic, with or without branching.

Percent sequence identity and Percent sequence similarity: used herein in comparisons of nucleic acid and/or amino acid sequences. Percent sequence identity and percent sequence similarity are used to refer to the percentage of sequence identity or sequence similarity between two or more nucleic acid sequences, or two or more polypeptide sequences. Sequence identity and similarity are typically measured using sequence analysis software with the default parameters specified therein (e.g., Sequence Analysis Software Package of the Genetics Computer Group (University of Wisconsin Biotechnology Center) or Sequence Alignment Software Library (University of Southern California). These software programs match similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

RNA polymerase: Refers to the DNA-dependent RNA polymerase enzyme involved in transcription, i.e. the process by which genes are transcribed to produce RNA. The DNA-dependent RNA polymerase enzyme of S. aureus exists in two forms: the Core-RNA polymerase (α2, β and β′ sub-units) and the holoenzyme (primary sigma factor plus core).

STAAU_R12 polypeptide or Staphylococcus aureus primary sigma factor or σ^{SA}: Generally refers to the primary sigma factor in S. aureus that is encoded by the plaC gene (GenBank™ acc. No. M63177). It includes polypeptides comprising the full-length amino acid sequence as set forth in SEQ ID NO: 2, and variants or fragments thereof such as those fragments set forth in SEQ ID NOs:3 to 5.

Variant and Homologue: As used herein, the term “variant(s)” and “homologue(s)” refers to a polynucleotide or polypeptide that differs from a reference polynucleotide or polypeptide, respectively, in size, sequence, structure or composition, but retains one or more of the biological activities of the reference (e.g. non-variant) polynucleotide or polypeptide. A typical variant or homologue of a polynucleotide differs in nucleotide sequence from a reference polynucleotide. Changes in the nucleotide sequence of the variant or homologue may or may not alter the amino acid sequence of a polypeptide encoded by the variant or homologous polynucleotide, compared to a reference polynucleotide. Nucleotide changes may result in amino acid substitutions, additions, deletions, and truncations in the polypeptide encoded by the variant or homologous sequence, or in the formation of fusion proteins, as discussed below. A typical variant or homologue of a polypeptide differs in amino acid sequence from a reference polypeptide. Generally, differences are limited so that the sequences of the reference polypeptide and the variant or homologue are closely similar overall and, in many regions, identical. A variant and reference polypeptide may differ in amino acid sequence by one or more substitutions, additions, deletions in any combination.

B) General Overview of the Invention

This invention relates, in part, to a specific binding interaction between a growth-inhibitory protein encoded by the genome of bacteriophage G1 and an essential S. aureus protein known as the primary sigma factor (σ^{SA}; also called herein “STAAU_R12”). STAAU_R12 was identified using the methodology described in detail in U.S. Pat. No. 6,376,652, and PCT International Application WO 00/32825, both incorporated herein by reference.

The phage G1 protein that binds to STAAU_R12 is referred to herein as G1ORF67. G1ORF67 (SEQ ID NO: 7) and a fragment thereof (SEQ ID NO: 8) constitute the first anti-sigma factors identified up to date for S. aureus primary sigma factor (σSA). This discovery serves as a basis for novel antimicrobial compounds, anti-infective compositions, methods of treatments, screening assays, etc. Indeed, the inventors have recognized the utility of the interaction in the development of antibacterial compounds. Specifically, the inventors have recognized that 1) STAAU_R12 is an important target for bacterial inhibition since it is essential for bacterial growth; 2) G1ORF67 or fragments or variants or functional mimetics thereof are useful for inhibiting bacterial growth; and 3) the interaction between STAAU_R12 (or fragments or variants thereof) and G1ORF67 may be used as a target for the screening and rational design of drugs or antibacterial compounds. In addition to methods of directly inhibiting STAAU_R12 activity, methods of inhibiting STAAU_R12 expression are also recognized attractive means for inhibiting bacterial activity.

Additionally, the inventors have identified a 195 amino acid protein from S. aureus bacteriophage Twort (herein called “TwortORF65”, SEQ ID NO: 10) which shares significant homology to G1ORF67. TwortORF65 is considered to be another anti-primary sigma factor, having similar STAAU_R12-binding activity and similar inhibitory activity as G1ORF67. Accordingly, all aspects of the invention relating to G1ORF67 also include corresponding aspects using TwortORF65 (or a fragment or variant thereof) and/or using any polypeptide (or a fragment or variant or homologue thereof) comprising a G1ORF67/TwortORF65-consensus sequence as set forth in SEQ ID NO: 12.

i) G1ORF67 Polynucleotides and Polypeptides

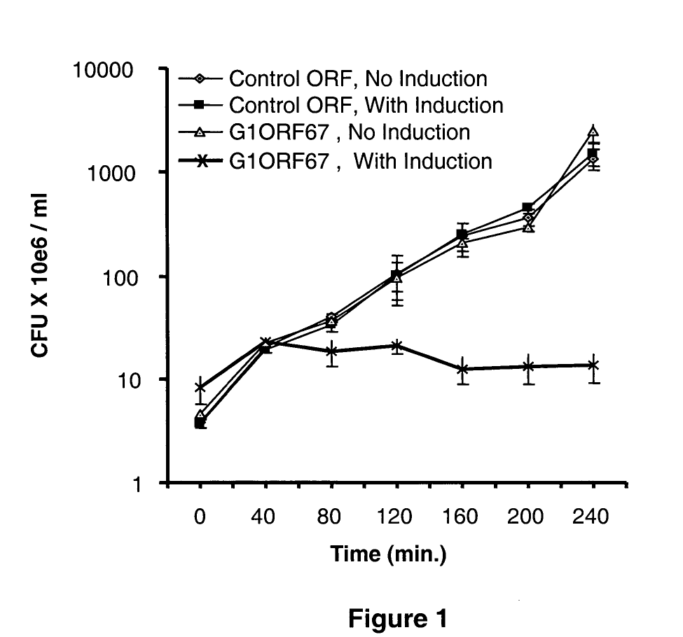

As it will be described hereinafter in the Exemplification section, the inventors have discovered, cloned and sequenced a growth inhibitory protein which is encoded by the genome of bacteriophage G1 and which has a bacteriostatic activity in S. aureus as shown in FIG. 1. This phage protein is referred herein as G1ORF67 (SEQ ID NO: 7).

Accordingly, the invention concerns an isolated, purified or enriched nucleic acid molecule comprising nucleotides 1-597 of SEQ ID NO:6, encoding G1ORF67. The invention also concerns an isolated, purified or enriched nucleic acid molecule comprising a polynucleotide fragment of at least 15, 25, 50, 75, 100, 150, 200, 300, 400, 500, 550 or more contiguous nucleotides of the bacteriophage nucleic acid sequence set forth in SEQ ID NO: 6 (G1ORF67). In addition, the invention concerns an isolated, purified or enriched nucleic acid molecule variant that has at least 50%, 60%, 70%, 75%, 80%, 85%, 90%, 95%, and even 100% sequence identity with (1) the polynucleotide set forth in SEQ ID NO:6, (2) a fragment of the polynucleotide set forth in SEQ ID NO:6, or (3) a nucleic acid sequence encoding a polypeptide as set forth in SEQ ID NO: 7, SEQ ID NO: 8 or with the complement thereof. The invention is further concerned with polynucleotides homologues that hybridize under stringent hybridization conditions with (1) the complement of the polynucleotide set forth in SEQ ID NO:6, (2) the complement of a fragment of the polynucleotide set forth in SEQ ID NO:6, (3) the complement of a nucleic acid sequence encoding the amino acid sequence set forth in SEQ ID NO:7, or (4) the complement of a nucleic acid sequence encoding the amino acid sequence set forth in SEQ ID NO:8. Preferred polynucleotides of the invention include the polynucleotide set forth in SEQ ID NO: 6, non-bacterial polynucleotides encoding a polypeptide that binds the Staphylococcus aureus primary sigma factor polypeptide set forth in SEQ. ID NO: 2, and non-bacterial polynucleotides encoding a polypeptide that inhibits growth of S. aureus.

A related aspect of the invention concerns isolated or purified bacteriophage polypeptides. In one embodiment, the bacteriophage polypeptide of the invention binds the Staphylococcus aureus primary sigma factor polypeptide set forth in SEQ ID NO: 2. In another embodiment, the bacteriophage polypeptide of the invention comprises amino acids 1-198 of SEQ ID NO:7 (G1ORF67). In a further embodiment, the bacteriophage polypeptide comprises a polypeptide fragment of at least 10, 20, 30, 40, 50, 75, 100, 125, 150 or more contiguous amino acids of the amino acid sequence set forth in SEQ ID NO: 7 (G1ORF67). In addition, the invention concerns a polypeptide variant that at least 50%, 60%, 70%, 75%, 80%, 85%, 90%, 95%, and even 100% sequence identity with (1) the amino acid sequence set forth in SEQ ID NO: 7, (2) the amino acid sequence set forth in SEQ ID NO: 8, or (3) a fragment of SEQ ID NO:7, wherein the variant binds the polypeptide set forth in SEQ ID NO: 2, and/or inhibiting growth of S. aureus. The invention is further concerned with polypeptides homologues encoded by polynucleotides that hybridize under stringent hybridization conditions with (1) the complement of the polynucleotide set forth in SEQ ID NO:6, (2) the complement of a fragment of the polynucleotide set forth in SEQ ID NO:6, (3) the complement of a nucleic acid sequence encoding the amino acid sequence as set forth in SEQ ID NO:7, or (4) the complement of a nucleic acid sequence encoding the amino acid sequence set forth in SEQ ID NO:8. Preferred polypeptides of the invention include the polypeptides comprising amino acids 1-198 of SEQ ID NO: 7 or amino acids 1-149 of SEQ ID NO: 8. Such polypeptides may be used into screening methods, diagnostic methods, and into methods for treating microbial infections and conditions associated with such infections as defined herein.

The invention also relates to vectors that comprise a polynucleotide or polynucleotides of the invention, host cells that are genetically engineered with vector(s) of the invention and the production of polypeptides of the invention by recombinant techniques. Accordingly, the invention also concerns a method for producing a G1ORF67 polypeptide, and fragments and variants thereof. This method comprises the steps of: (i) providing a cell transformed with a nucleic acid sequence encoding a G1ORF67 polypeptide, or a fragment or variant thereof, positioned for expression in the cell; (ii) culturing the transformed cell under conditions suitable for expressing the nucleic acid; (iii) producing said a G1ORF67 polypeptide; and optionally, (iv) recovering the G1ORF67 polypeptide produced.

Once the recombinant protein is expressed, it is isolated by using any suitable purification technique such as ammonium sulfate or ethanol precipitation, acid or urea extraction, anion or cation exchange chromatography, phosphocellulose chromatography, hydrophobic interaction chromatography, affinity chromatography, hydroxylapatite chromatography, and lectin chromatography. Once isolated, the recombinant protein can, if desired, be purified further. Methods and techniques for expressing recombinant proteins and foreign sequences in prokaryotes and eukaryotes are well known in the art and will not be described in more detail. One can refer, if necessary, to Joseph Sambrook, David W. Russell, Joe Sambrook, Molecular Cloning: A Laboratory Manual, (2001) Cold Spring Harbor Laboratory Press. Those skilled in the art of molecular biology will understand that a wide variety of expression systems may be used to produce the recombinant protein. The precise host cell used is not critical to the invention. G1ORF67 polypeptides of the invention, particularly short G1ORF67 fragments, may also be produced by chemical synthesis. These general techniques of polypeptide expression and purification can also be used to produce and isolate useful G1ORF67 fragments or analogs, as described herein.

ii) TwortORF65 and G1ORF671 TwortORF65-Consensus Polynucleotides and PolypePtides

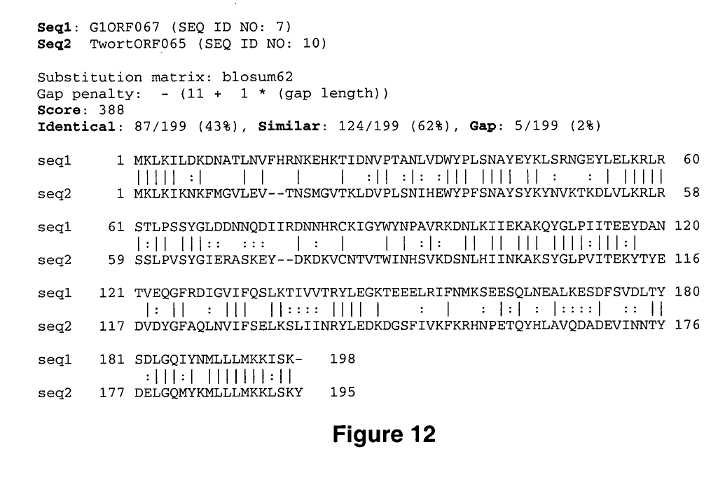

As indicated hereinbefore, the inventors have identified TwortORF65 (SEQ ID NO: 10), considered to be an additional anti-primary sigma factor, potentially having similar STAAU_R12-binding activity and inhibitory activity as G1ORF67. Indeed, as shown in FIG. 12, this bacteriophage protein shares 43% identity and 62% similarity with the amino acid sequence of G1ORF67 (61% identity at the nucleotide level). In the C-terminal portion corresponding to amino acid 50-198 of G1ORF67, the region of G1ORF67 shown to be important for binding STAAU_R12, the two bacteriophage proteins share 43% identity and 64% similarity at the amino acid level (61% identity at the nucleotide level). Furthermore, as with G1ORF67, TwortORF65 has a growth inhibitory effect on S. aureus (not shown). Finally, the genetic maps (i.e. genes arrangement in the genome) of bacteriophages G1 and TWORT are almost identical. Taken together, there results suggest that both phage proteins have the same function (not shown).

Accordingly, the invention encompasses all aspects of the invention relating to G1ORF67 polypeptides and nucleotides but substituting TwortORF65 polypeptides and nucleotides instead. The invention further encompasses all aspects of the invention relating to G1ORF67 but substituting any polypeptide (or a fragment or variant or homologue thereof) comprising a G1ORF67/TwortORF65-consensus amino acid sequence as set forth in SEQ ID NO: 12 instead of G1ORF67-related polypeptide, or by substituting any polynucleotide (or a fragment or variant thereof) comprising a G1ORF67/TwortORF65 consensus nucleotide sequence as set forth in SEQ ID NO: 11 instead of G1ORF67-related nucleotide.

More specifically, another aspect of the invention concerns an isolated, purified or enriched nucleic acid molecule comprising nucleotides 1-588 of SEQ ID NO: 9 encoding TwortORF65, or comprising nucleotides 1-585 of SEQ ID NO: 11 (encoding a G1ORF67/TwortORF65 consensus amino acids sequence). The invention also concerns an isolated, purified or enriched nucleic acid molecule comprising a polynucleotide fragment of at least 15, 25, 50, 75, 100, 150, 200, 300, 400, 500, 550 or more contiguous nucleotides of the bacteriophage nucleic acid sequence set forth in SEQ ID NO: 9 (TwortORF65) or SEQ ID NO: 11. In addition, the invention concerns an isolated, purified or enriched nucleic acid molecule variant that has at least 50%, 60%, 70%, 75%, 80%, 85%, 90%, 95%, and even 100% sequence identity with (1) the polynucleotide set forth in SEQ ID NO:9 or SEQ ID NO: 11, (2) a fragment of the polynucleotide set forth in SEQ ID NO:9 or SEQ ID NO: 11, or (3) a nucleic acid sequence encoding a polypeptide as set forth in SEQ ID NO: 10 or SEQ ID NO: 12; or with the complement thereof. The invention is further concerned with polynucleotides homologues that hybridize under stringent hybridization conditions with (1) the complement of the polynucleotide set forth in SEQ ID NO: 9 or SEQ ID NO: 11, (2) the complement of a fragment of the polynucleotide set forth in SEQ ID NO:9 or SEQ ID NO: 11, or (3) the complement of a nucleic acid sequence encoding the amino acid sequence set forth in SEQ ID NO:10 or SEQ ID NO: 12.

A related aspect of the invention concerns isolated or purified bacteriophage polypeptides from phage TWORT and from others bacteriophages. In one embodiment, the bacteriophage polypeptide of the invention comprises amino acids 1-195 of SEQ ID NO:10 (TwortORF65). In another embodiment, the bacteriophage polypeptide of the invention comprises amino acids 1-194 of SEQ ID NO:12 (G1ORF67/TwortORF65 consensus amino acids sequence). In a further embodiment, the bacteriophage polypeptide comprises a polypeptide fragment of at least 10, 20, 30, 40, 50, 75, 100, 125, 150 or more contiguous amino acids of the amino acid sequence set forth in SEQ ID NO: 10 or SEQ ID NO:12. In addition, the invention concerns a polypeptide variant that at least 50%, 60%, 70%, 75%, 80%, 85%, 90%, 95%, and even 100% sequence identity with (1) the amino acid sequence set forth in SEQ ID NO: 10 or in SEQ ID NO:12, (2) amino acids 48 to 194 as set forth in SEQ ID NO: 10 or SEQ ID NO:12, or (3) a fragment of SEQ ID NO:10 or of SEQ ID NO:12, wherein the variant binds the polypeptide set forth in SEQ ID NO: 2, and/or inhibiting growth of S. aureus. The invention is further concerned with polypeptides homologues encoded by polynucleotides that hybridize under stringent hybridization conditions with (1) the complement of the polynucleotide set forth in SEQ ID NO:9 or in SEQ ID NO:11, (2) the complement of a fragment of the polynucleotide set forth in SEQ ID NO:9 or in SEQ ID NO:11, or (3) the complement of a nucleic acid sequence encoding the amino acid sequence as set forth in SEQ ID NO:10 or in SEQ ID NO:11. Preferred polypeptides of the invention include the polypeptides comprising amino acids 1-195 of SEQ ID NO: 10, amino acids 48-194 of SEQ ID NO: 10, amino acids 1-194 of SEQ ID NO:12, or amino acids 48-194 of SEQ ID NO:12. Such polypeptides may be used into screening methods, diagnostic methods, and into methods for treating microbial infections and conditions associated with such infections as defined herein for G1ORF67.

The invention also relates to vectors that comprise a polynucleotide or polynucleotides of the invention, host cells that are genetically engineered with vector(s) of the invention and the production of polypeptides of the invention by recombinant techniques. Accordingly, the invention also concerns a method for producing a TwortORF65 polypeptide or a G1ORF67/TwortORF65-consensus polypeptide, and fragments and variants thereof, as described hereinbefore for G1ORF67.

iii) STAAU_R12 or S. aureus Primary Sigma Factor is the Bacterial Target of G1ORF67

As it will be described with more details hereinafter, G1ORF67 binds to an essential S. aureus protein known as the primary sigma factor (σ^{SA}; also called herein STAAU_R12). The identified STAAU_R12 polypeptide was compared with all other sequences in the public domain databases and results revealed that this protein is well conserved in prokaryotes, including B. subtilis (79% identity) and E. coli (σ⁷⁰; 61% identity).

Interestingly, as shown hereinafter in the examples, the present inventors have produced STAAU_R12 fragments that retain the abilityto interact with G1ORF67. Accordingly, another aspect of the invention features an isolated or purified bacterial polypeptide fragment of the S. aureus primary sigma factor (σ^{SA}) set forth in SEQ ID NO: 2, this bacterial polypeptide fragment comprising a bacteriophage polypeptide binding domain. The bacterial polypeptide domain may comprise as few as 5 amino acids (preferably from about 25 to about 245 amino acids) and up to 367 amino acids. Preferably, the bacteriophage polypeptide binding domain binds a polypeptide from bacteriophage G1, more preferably the bacteriophage polypeptide set forth in SEQ ID NO: 7 (G1ORF67) and/or the bacteriophage polypeptide consisting of amino acids 50-198 of G1ORF67 (SEQ ID NO: 8). In a preferred embodiment, the bacteriophage polypeptide binding domain comprises an amino acid sequence selected from the group consisting of amino acids 127-368 of ₀SA (SEQ ID NO: 3), amino acids 294-368 of σ^{SA} (SEQ ID NO: 4) and amino acids 294-360 of σ^{SA} (SEQ ID NO: 5). Such bacterial polypeptide fragment(s) may be used into screening methods and diagnostic methods as defined herein.

Of course, other fragments or variants of STAAU_R12 can be cloned using technologies known in the art (see hereinbefore), the binding interaction with a G1ORF67 polypeptide could be tested by affinity chromatography, by using a yeast two-hybrid assay, by Fluorescence resonance energy transfer as exemplified hereinafter, or by using any other suitable protein-protein interaction assay. Fragments or variants of STAAU_R12 can also be prepared by chemical synthesis and/or partial proteolysis.

In a related aspect, the invention concerns methods for inhibiting bacterial growth, preferably S. aureus growth. In one embodiment, the method comprises contacting a bacterium with an antibacterial compound that specifically binds to a bacteriophage polypeptide binding domain of Staphylococcus aureus primary sigma factor polypeptide. Preferably, the binding domain binds the G1ORF67 polypeptide. More preferably, the binding domain comprises an amino acid sequence selected from the group consisting of:

(i) the amino acid sequence set forth in SEQ ID NO: 3;

(ii) the amino acid sequence set forth in SEQ ID NO: 4;

(iii) the amino acid sequence set forth in SEQ ID NO: 5;

(iv) a fragment of (i) or (ii) containing said bacteriophage polypeptide binding domain; and (v) a variant having at least 95% sequence identity with one of (i), (ii), (iii), and (iv), and containing a domain that is bound by said bacteriophage polypeptide.

In another embodiment, the method for inhibiting bacterial growth comprises contacting the bacterium with an antibacterial compound, and/or an anti-primary sigma factor and/or an anti-infective composition as defined hereinbefore. The contacting may be performed in vitro (in biochemical and/or cellular assays), in vivo in a non-human animal and/or in vivo in mammals, including humans. According to a related aspect the invention concerns a method for treating or preventing a bacterial infection in a mammal, comprising administering to the mammal a therapeutically effective or prophylactic effective amount of an antibacterial compound and/or an anti-primary sigma factor and/or an anti-infective composition as defined herein.

A further aspect of the invention concerns a method for inhibiting in S. aureus, σ^{SA}-dependent RNA polymerase activity, and more particularly a σ^{SA}-dependent RNA polymerase activity which is inhibitable by a bacteriophage polypeptide. In one embodiment, the method comprises contacting a S. aureus bacterium with an antibacterial compound that specifically binds to a bacteriophage polypeptide binding domain of Staphylococcus aureus primary sigma factor polypeptide.

In a related aspect, the invention also concerns an isolated or purified enriched antibody that specifically binds to the S. aureus primary sigma factor polypeptide, or a fragment or variant of the S. aureus primary sigma factor polypeptide as defined hereinabove or to a G1ORF67 or to a TwortORF65 polypeptide, fragment or variant as defined herein. Also included in the present invention are hybridomas expressing such antibodies. Antibodies generated against the polypeptides of the invention may be obtained by using any suitable technique known in the art. Such antibodies could be useful for treatment of infections or as a research tools to purify the polypeptides or polynucleotides by affinity chromatography for instance.

iv) Antimicrobial Compositions and Methods of Treatment

Based on the results presented hereinafter in the Exemplification section, skilled artisans will recognize that the present inventors are the first ones to discover inhibitors (anti-sigma factors) of the primary sigma factor in Staphylococcus aureus.

The present inventors have clearly demonstrated, both in biochemical and in cellular assays, that: (i) G1ORF67 has a bacteriostatic effect on Staphylococcus aureus; (ii) both σ^{SA} DNA binding activity and σ^{SA} mediated transcriptional activity are negatively affected by G1ORF67 and its fragment; (iii) G1ORF67 and its fragment physically interact with STAAU_R12 (σ^{SA}); (iv) it is the specific interaction between σ^{SA} and G1ORF67 (and/or its fragment) which negatively modulate σ^{SA} function; and (v) RNA polymerase activity of Staphylococcus aureus is σ^{SA}-dependent and that activity may be inhibited by a bacteriophage polypeptide such as G1ORF67. Additionally, TwortORF65 is likely another anti-primary sigma factor, having binding and inhibitory activity similar to that of G1ORF67.

Therefore, the invention features an anti-sigma factor which inhibits or blocks the physiological function (i.e. the biochemical and/or cellular activity) of S. aureus primary sigma factor (σ^{SA}), or the activity of a biologically active fragment or variant thereof. The physiological function of the S. aureus primary sigma factor (σ^{SA}), fragment or variant that may be inhibited by the anti-primary sigma factor of the invention includes, but is not limited to transcriptional activation, binding to S. aureus Core-RNA polymerase, and binding to DNA. In a preferred embodiment, the inhibition of function is caused by direct binding of the anti-primary sigma factor to the σ^{SA}, fragment or variant.

According to another aspect, the invention features an antibacterial compound. In one preferred embodiment, the antibacterial compound inhibits in S. aureus, σSA-dependent RNA polymerase activity. In another embodiment, the antibacterial compound inhibits or blocks the DNA binding activity of a S. aureus primary sigma factor (σ^{SA}) or of a biologically active fragment or variant thereof. Yet in a further embodiment, the antibacterial compound inhibits interaction between: (i) S. aureus primary sigma factor (sA) or of a biologically active fragment or variant thereof; and (ii) S. aureus Core-RNA polymerase. Preferably, the inhibition is caused by direct binding of the antibacterial compound to the σ^{SA}, fragment or variant.

Yet another aspect of the invention concerns an antibacterial compound having a bactericidal or bacteriostatic effect on Staphylococcus aureus, this antibacterial compound binding to a polypeptide comprising the amino acid sequence of SEQ ID NO: 2 (σ^{SA}). Preferably, the binding of the antibacterial compound to the polypeptide is specific.

In one embodiment, the anti-primary sigma factor and/or the antibacterial compound defined hereinbefore binds a bacteriophage binding domain of S. aureus primary sigma factor, the bacteriophage binding domain comprising an amino acid sequence selected from the amino acid sequence set forth in SEQ ID NO: 3; the amino acid sequence set forth in SEQ ID NO: 4; and the amino acid sequence set forth in SEQ ID NO: 5. Preferably, the anti-primary sigma factor and/or the antibacterial compound mimics the inhibitory activity, and/or the bactericidal or bacteriostatic effect, of the bacteriophage polypeptide comprising amino acids 1-198 of SEQ ID NO: 7 or amino acids 50-198 of SEQ ID NO: 8. The anti-primary sigma factor and/or the antibacterial compound may also mimics the inhibitory activity, and/or the bactericidal or bacteriostatic effect, of the bacteriophage polypeptide comprising amino acids 1-195 of SEQ ID NO: 10, amino acids 48-194 of SEQ ID NO: 10 or of a polypeptide comprising amino acids 48-194 of SEQ ID NO: 12.

The anti-primary sigma factor and the antibacterial compounds defined hereinbefore may be a small molecule, a peptidomimetic compound, a peptide, or a polypeptide. Suitable polypeptides include bacteriophage proteins such as SEQ ID NO: 7 (G1ORF67), SEQ ID NO: 8 (AA 50-198 of G1ORF67), and biologically active fragments and variants thereof binding to the polypeptide set forth in SEQ ID NO: 2 and/or inhibiting S. aureus growth. In another embodiment, the anti-primary sigma factor may is selected amongst polypeptides comprising amino acids 1-195 of SEQ ID NO: 10, amino acids 48-194 of SEQ ID NO: 10 and polypeptides comprising amino acids 48-194 of SEQ ID NO: 12, and biologically active fragments and variants thereof binding to the polypeptide set forth in SEQ ID NO: 2 and/or inhibiting S. aureus growth. Small molecules according to the invention include organic and inorganic chemical entities purified from natural sources (e.g. plants, fungi, etc) or synthesized in a laboratory. Potential small molecules also include chemical compounds inhibiting G1ORF67—S. aureus primary sigma factor interaction, chemical compounds inhibiting TwortORF65—S. aureus primary sigma factor interaction, and compounds identified by a screening method as defined hereinafter. Peptidomimetic compounds include but are not limited to biomimetics, and functional mimetics of the natural G1ORF67 or its fragments as defined herein, or of the natural TwortORF65. Of course, peptidomimetic compounds, peptides, and polypeptides according to the invention may be modified according to methods well known it the art to make them less immunogenic to individuals, to increase their solubility or for any other useful purpose. Furthermore, some methods of screening for such small molecules and peptidomimetics are provided hereinafter.

More preferably, the anti-primary sigma factor and the antibacterial compound defined hereinbefore have at least one, even more preferably two, three or all, of the following biochemical and/or cellular activities:

inhibition of DNA binding activity of σ^{SA}, of its fragment and/or its variant;

inhibition of the binding between: (i) S. aureus primary sigma factor (CSA), its fragment and/or variant; and (ii) S. aureus Core-RNA polymerase;

inhibition of S. aureus primary sigma factor (SAy)-mediated transcriptional activation; and

inhibition of S. aureus holoenzyme RNA polymerase activity or function.

Skilled artisans will also recognize that the antibacterial compounds and/or the anti-primary sigma factor as described herein may serve as an active ingredient in a therapeutic or anti-infective composition for therapeutic or prophylactic purposes. Thus, it will be understood that another aspect of the invention described herein, includes the compounds of the invention in combination with a pharmaceutically acceptable carrier or diluent. Of course, more than one active compound of the present invention could be combined, with or without existing classes of antibiotics such as sulfonamides, beta-lactams, tetracyclines, chloramphenicol, aminoglycosides, macrolides, glycopeptides, streptogamins, quinolones, oxazolidinones, and lipopeptides. Therefore, the present invention provides for anti-infective compositions comprising a therapeutically effective amount of an antibacterial compound and/or an anti-primary sigma factor as described herein in combination with a pharmaceutically acceptable carrier or excipient. Such carriers include, but are not limited to saline, buffered saline, dextrose, water, glycerol, ethanol, and combinations thereof. The pharmaceutical compositions may be administered in any effective, convenient manner including, for instance, administration by topical, oral, anal, vaginal, intravenous, intraperitoneal, intramuscular, subcutaneous, intranasal or intradermal routes among others.

In therapy or as a prophylactic, the active compound may be administered to an individual as an injectable composition, for example as a sterile aqueous dispersion, preferably isotonic. Alternatively the composition may be formulated for topical application for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation. Alternative means for systemic administration include transmucosal and transdermal administration using penetrants such as bile salts or fusidic acids or other detergents. In addition, if a polypeptide or other compounds of the present invention can be formulated in an enteric or an encapsulated formulation, oral administration may also be possible. Administration of these compounds may also be topical and/or localized, in the form of salves, pastes, gels, and the like.

For administration to mammals, and particularly humans, it is expected that the daily dosage level of the active compound will be from 0.01 mg/kg to 10 mg/kg, typically around 1 mg/kg. The physician in any event will determine the actual dosage that will be most suitable for an individual and will vary with the age, weight and response of the particular individual. The above dosages are exemplary of the average case. There can, of course, be individual instances where higher or lower dosage ranges are merited, and such are within the scope of this invention.

In accordance with related aspects, the invention concerns methods for inhibiting bacterial growth (preferably S. aureus growth), methods for inhibiting σ^{SA}-dependent RNA polymerase activity, and methods for treating or preventing a bacterial infection in a mammal thereby inhibiting or ablating growth and/or metabolic activity of a bacterium or bacterial population.

In one embodiment, the method comprises contacting a bacterium with an antibacterial compound that specifically binds to a bacteriophage polypeptide binding domain of Staphylococcus aureus primary sigma factor polypeptide. In one embodiment, the bacteriophage polypeptide binding domain comprises an amino acid sequence selected from the group consisting of:

(i) the amino acid sequence set forth in SEQ ID NO: 3;

(ii) the amino acid sequence set forth in SEQ ID NO: 4;

(iii) the amino acid sequence set forth in SEQ ID NO: 5;

(iv) a fragment of (i) or (ii) containing said bacteriophage polypeptide binding domain; and

(v) a variant having at least 95% sequence identity with one of (i), (ii), (iii), and (iv), and containing a domain that is bound by said bacteriophage polypeptide.

In another embodiment, the method for inhibiting bacterial growth comprises contacting the bacterium with an antibacterial compound, and/or an anti-primary sigma factor and/or an anti-infective composition as defined hereinbefore. The contacting may be performed in vitro (e.g. biochemical and/or cellular assays and techniques), in vivo in a non-human animal and/or in vivo in humans.

In a preferred embodiment, the method for inhibiting σSA-dependent RNA polymerase activity is performed in S. aureus. Inhibition is obtained by contacting the RNA polymerase, preferably in presence also of σ^{SA}, with a compound having a suitable level of inhibitory activity, such as the antibacterial compounds defined hereinbefore. Preferably, the σ^{SA}-dependent RNA polymerase activity is inhibitable by a bacteriophage polypeptide. The compounds may be contacted in vitro or in vivo, whether for the purpose of screening assays and for the prevention or treatment of S. aureus infections.

According to a related aspect, the invention concerns a method for treating or preventing a bacterial infection in a mammal, comprising administering to the mammal a therapeutically effective or prophylactic effective amount of an antibacterial compound and/or an anti-primary sigma factor and/or an anti-infective composition as defined hereinbefore. In one embodiment, the method for treating or preventing a bacterial infection in a host (preferably a mammal and more preferably a human) comprises administering to the host a therapeutically effective or prophylactic effective amount of an antibacterial compound and/or an anti-primary sigma factor and/or an anti-infective composition as defined hereinbefore. In a preferred embodiment, the antibacterial compound and/or an anti-primary sigma factor bind specifically to a bacteriophage polypeptide binding domain of a S. aureus primary sigma factor (σ^{SA}). Examples of such binding domains include but is not limited to SEQ ID NO: 3 (AA 127-368), SEQ ID NO: 4 (AA 294-368), SEQ ID NO: 5 (AA 294-360), and fragments or variants thereof.

Preferably the bacterium is from Staphylococcus aureus strains, including antibiotics resistant strains, but it is conceivable that the antibacterial compounds and/or an anti-primary sigma factor and/or an anti-infective compositions according to the present invention possess a broader spectrum of inhibitory activity, including antibacterial activities against one or more Gram positive bacteria (including but not limited to Staphylococcus aureus, Staphylococcus saprophyticus, Staphylococcus epidermis, Streptococcus agalactiae, Streptococcus faecium, Streptococcus durans, Streptococcus faecalis, Enterococcus faecalis, Bacillus subtilis, Streptococcus pneumoniae, Streptococcus pyogenes, mycobacterium), Gram negative bacteria (including but not limited to Haemophilus influenzae, Haemophilus aegyptius, Haemophilus parainfluenzae, Haemophilus ducreyi, Moxarella catarrhalis, Escherichia coli, Listeria monocytgens, Samonella, Shigella dysenteriae, Neisseria gonorrhea, Chlamydia pneumoniae, Legionella spp., Helicobacter pylon), an archaeon, including but not limited to Archaebacter, and a unicellular or filamentous eukaryote (including but not limited to a protozoan, a fungus, a member of the genus Saccharomyces, Kluyveromyces, or Candida, and a member of the species Saccharomyces ceriviseae, Kluyveromyces lactis, or Candida albicans). Therefore, the invention encompasses therapeutic or prophylactic methods against many diseases caused by or related to bacterial infection, including but not limited to otitis, conjunctivitis, pneumonia, bacteremia, meningitis, sinusitis, pleural empyema and endocarditis, and most particularly meningitis, such as, for example, infection of cerebrospinal fluid. In such methods, an effective therapeutic or prophylactic amount of an antibacterial compound and/or an anti-primary sigma factor and/or an anti-infective composition as defined hereinbefore, is administered to a mammal in an amount sufficient to provide a therapeutic effect and thereby prevent or threat the infection of the mammal. Exact amounts can be routinely determined by one skilled in the art and will vary depending on several factors, such as the particular bacterial strain involved and the particular antibacterial compound used.

v) Methods of Screening and Making Antibacterial Compounds

According to another aspect, the invention features screening methods. In one embodiment, the method comprises the steps of:

(a) contacting a Staphylococcus aureus primary sigma factor polypeptide comprising a bacteriophage binding domain with a test compound in the presence of a bacteriophage polypeptide that specifically binds to the bacteriophage binding domain; and

(b) determining whether the test compound inhibits binding of the bacteriophage polypeptide to the bacteriophage binding domain.

Preferably, the bacteriophage polypeptide is a polypeptide of bacteriophage G1. More preferably, the bacteriophage polypeptide comprises amino acids 1-198 of SEQ ID NO: 7, or amino acids 50-198 SEQ ID NO: 8. The invention also encompasses fragments or variants of SEQ ID NO: 7 and/or SEQ ID NO: 8, with similar binding activity than that the original bacteriophage polypeptide(s). In another embodiment, the bacteriophage polypeptide is a polypeptide of bacteriophage Twort, preferably a bacteriophage polypeptide comprising amino acids 1-195 of SEQ ID NO: 10, or amino acids 48-194 SEQ ID NO: 10 or a fragment or variant or homologue thereof.

In another embodiment, the method comprises the steps of:

(a) contacting (i) a first polypeptide binding domain, (ii) a second polypeptide binding domain and (iii) at least one test compound, wherein the first and second polypeptide binding domains bind specifically with each other,

wherein the first polypeptide binding domain comprises an amino acid sequence selected from the group consisting of: the amino acid sequence set forth in SEQ ID NO: 3; the amino acid sequence set forth in SEQ ID NO: 4; and the amino acid sequence set forth in SEQ ID NO: 5;

wherein the second polypeptide binding domain comprises the amino acid sequence set forth in SEQ ID NO: 7 or SEQ ID NO: 8; and

(b) determining whether said at least one test compound inhibits binding between said first and second polypeptide binding domains.

The determination step may comprises a directly detectable (e.g., an isotope or a fluorophore) or indirectly detectable (e.g., an enzyme activity allowing detection in the presence of an appropriate substrate) measurement. The determination step may comprise a measurement by various techniques such as Fluorescence Resonance Energy Transfer (FRET), fluorescence polarization, surface plasmon resonance, scintillation proximity assay, biosensor assay, and phage display.

In a preferred embodiment, a library of test compounds is used for screening. Libraries generally include collections of 100 compounds, preferably of 1000, still more preferably 5000, still more preferably 10,000 or more, and most preferably of 50,000 or more compounds.

A “test compound” as used herein, is any compound with a potential to modulate the activity or function of S. aureus STAAU_R12 polypeptide (σ^{SA}). Non-limiting examples of test compounds include small molecules, mimetics compounds, antibodies, nucleic acids molecules, peptides, and fragments or derivatives of a bacteriophage inhibitor protein. Preferred compounds include small molecules that bind to and occupy a binding site of σ^{SA}, thereby preventing binding of σ^{SA} to DNA or to cellular binding molecules (e.g. Core-RNA polymerase). The small molecule may be organic or inorganic, natural or synthetic and it preferably has a molecular mass of less than 3000 Daltons, more preferably less than 2000 or 1500, still more preferably less than 1000, and most preferably less than 600 Daltons.

As used herein, the term “measuring” or “determining” a “binding interaction” refers to the use of an assay permitting determination of the existence and/or quantization of a physically association between two molecules. For instance, the equilibrium binding concentration of a polypeptide that specifically binds another is generally in the range of about 1 mM or lower, more preferably 1 uM or lower, preferably 100 nM or lower, 10 nM or lower, 1 nM or lower, 100 pM or lower, and even 10 pM or lower. Decrease or inhibition of binding may be measured directly or indirectly under one set of conditions relative to another set of reference conditions. Preferably, decrease or inhibition of binding is achieved if at least 10% and more preferably 20%, 40%, 50%, 75%, 90%, 95% or even as much as 100% (i.e., no detectable interaction) less binding is measured when compared to binding under reference conditions. Under some circumstances, binding could be measured by coupling one molecule to a surface or support such as a membrane, a microtiter plate well, or a microarray chip, and the binding of a second molecule could be monitored by any number of means including but not limited to optical spectroscopy, fluorometry, and radioactive label detection. Techniques for coupling or immobilizing molecules such as proteins or polypeptides on suitable matrices (e.g. beads, microtitre plates, chips, etc) are well-known in the art and may require the use of fusion-proteins in order to add a domain (e.g. glutathione-S-transferase (GST), biotin, antibody-reactive domain, 6X-Histitide, calmodulin binding protein, etc) that allows one or both of the proteins to be bound to a matrix.

Preferably also, the screening methods of the invention further comprise one or more steps such as:

measuring the ability of the test compound selected to inhibit or block DNA binding by the σ^{SA} polypeptide, fragment or variant;

measuring the ability of the test compound selected to inhibit or block binding between: (i) the S. aureus primary sigma factor polypeptide; and (ii) S. aureus Core-RNA polymerase;

measuring the ability of the test compound selected to inhibit or block S. aureus RNA polymerase activity;

measuring bactericidal and/or bacteriostatic activity of the test compound selected.

Measurement of the biological activities of test compounds may be made directly or indirectly. As mentioned previously, biological activity may include simple binding to other factor(s) (polypeptides or otherwise), including compounds, substrates, and interacting proteins. Biological activity also includes any standard biochemical measurement of a protein or enzyme such as conformational changes, phosphorylation status or any other feature of the protein that can be measured with techniques known in the art.

The following Exemplification section provides numerous examples of methods and techniques for assessing the biochemical, cellular and/or physiological activity or functions of STAAU_R12 (σ^{SA}) and of actual or potential anti-primary sigma factors and antibacterial agents/compounds according to the invention. Suitable methods, techniques and assays include but are not limited to: (i) DNA binding assays (e.g. gel shift assay, TR-FRET); transcription or RNA synthesis assays; protein-protein interaction assays (e.g. yeast two-hybrid, TR-FRET, surface plasmon resonance, fluorescence polarization, phage display, protein gel shift assay, gel filtration, BiaCore™, kinase protection assay, crystal structure determination; isothermal titration microcalorimetry (ITC)); and bacterial growth inhibition assays (e.g. MIC). The present invention also encompasses biochemical in vitro screening methods for evaluating directly, in the absence of bacteriophage proteins, ability of test compounds to inhibit Staphylococcus aureus primary sigma factor activity or function, and thereby inhibit RNA synthesis. The above mentioned assays may thus also be practiced in the absence of bacteriophage proteins.

In a preferred embodiment, the screening methods of the invention are “high throughput method of screening” which means that they allow the evaluation or screening of a large plurality of compounds, rather than just one or a few compounds. Preferably the methods of screening according to the invention can be used to conveniently test at least 100, more preferably at least 1000, still more preferably at least 10,000, and most preferably at least 100,000 different compounds, or even more per day. In an even more preferred embodiment, the method of screening is amenable to automated, cost-effective high throughput screening on libraries of compounds for lead development.

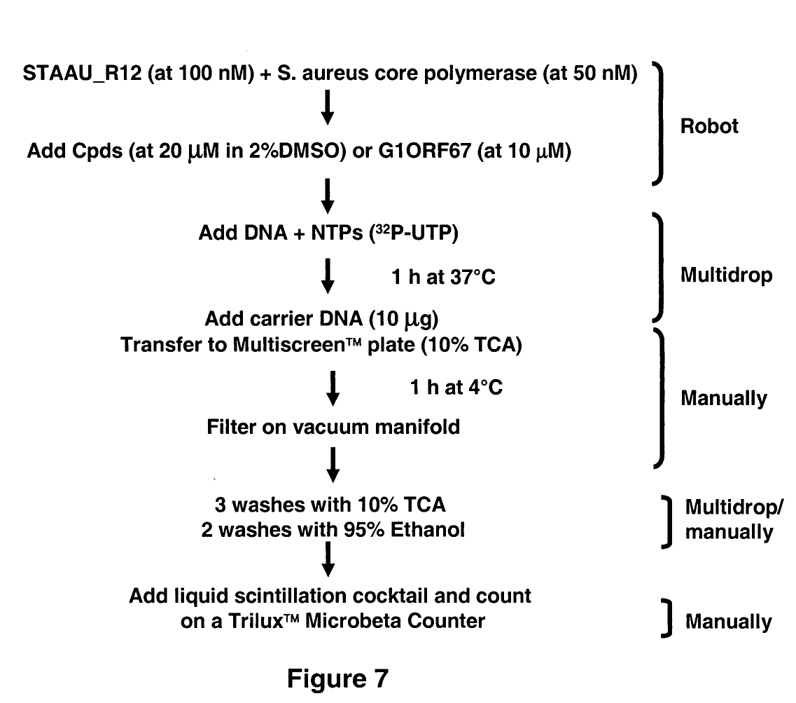

As mentioned previously, Staphylococcus aureus primary sigma factor may be used directly (without any bacteriophage protein) in biochemical in vitro screening assay. FIG. 7 shows a preferred embodiment of an automated, cost-effective high throughput screening assay for evaluating ability of a single or of a library of test compounds to inhibit STAAU_R12 (σ^{SA}) and thereby inhibit RNA synthesis (See also the Exemplification section: In vitro transcription and TCA precipitation for more details on this assay).

The assays and screening methods described herein may be used as initial or primary screens to detect promising lead compounds for further development. The same assays may also be used in a secondary screening assay to measure the activity of test compounds. Often, lead compounds will be further assessed in additional, different screens. This invention also includes secondary screens which may involve biological assays utilizing S. aureus strains or other suitable bacteria.

Tertiary screens may involve the study of the effect of the compound in an animal. Accordingly, it is within the scope of this invention to further use an anti-primary sigma factor or antibacterial compound identified as described herein in an appropriate animal model. For example, an antibacterial compound or anti-primary sigma factor identified as described herein can be used in an animal model to determine its efficacy, toxicity, or side effects of treatment. Alternatively, a compound or factor identified as described herein can be used in an animal model to determine the mechanism of action of such factor or compound. Furthermore, this invention pertains to uses of novel compounds and factors identified by the above-described screening assays for treatment (e.g. bacterial infections), as described hereinbefore.

In a related aspect, the invention features a method of making an antibacterial compound, the method comprising the steps of:

identifying a compound which interacts with a S. aureus primary sigma factor polypeptide by carrying out a screening method as defined previously; and

synthesizing or purifying the compound identified, preferably in an amount sufficient to provide a therapeutic or prophylactic effect when administered to an organism infected by S. aureus.

In a further embodiment, the method of making an antibacterial compound further includes a scaling-up of the preparation for synthesizing or purifying of the identified compound. In yet another embodiment of this method, the pharmaceutical composition prepared comprises a variant, derivative or homologue (e.g. structurally related molecule) of the compound identified.

vi) Diagnostic Assays

The invention further provides diagnostic assays and methods for diagnosing in a mammal an infection with S. aureus and/or for detecting diseases and conditions associated with such microbial infections. In one embodiment, the diagnostic assay detects the presence, expression and/or activity a S. aureus primary sigma factor polypeptide. Preferably, the method of diagnosing in a mammal, preferably in a human, an infection with S. aureus, comprises detecting binding between: (i) a first polypeptide binding domain derived from a S. aureus primary sigma factor polypeptide comprising SEQ ID NO: 2; and (ii) a second polypeptide domain derived from a bacteriophage polypeptide binding to the σ^{SA} protein. According to this method, the first and second domains are selected such that they bind to each other, and such that this binding is detectable. In a preferred embodiment, the first domain is a bacteriophage polypeptide binding domain. Suitable examples include polypeptides comprising an amino acid sequence selected from the group consisting of amino acids 127-368 of GSA (SEQ ID NO: 3), amino acids 294-368 of σ^{SA} (SEQ ID NO: 4) and amino acids 294-360 of σ^{SA} (SEQ ID NO: 5). Preferably also, the second polypeptide domain is from bacteriophage G1, more preferably it is a bacteriophage polypeptide comprising an amino acid sequence as set forth in SEQ ID NO: 7 or SEQ ID NO: 8. The second polypeptide domain may also be a polypeptide from bacteriophage Twort, more preferably a bacteriophage polypeptide comprising amino acids 1-195 of SEQ ID NO: 10, or amino acids 48-194 of SEQ ID NO: 10, or any polypeptide comprising amino acids 1-194 of SEQ ID NO: 12.

The first domain (derived from σ^{SA}) may be obtained from a putatively infected and/or infected individual's bodily materials, i.e. any material susceptible to contain σ^{SA} polypeptides or fragments, including but not limited to cells, tissues, waste or fluids.

Furthermore, the first and second domains could be part of a diagnostic kit for diagnosing an infective disease. Also, any of the bacteriophage polypeptide, fragment or variant defined herein before, exhibiting the ability of binding to the σ^{SA} protein could potentially be used in processes for diagnosing S. aureus bacterial infections.

vii) Sequence Databases and Sequences in a Tangible Medium

Polynucleotide and polypeptide sequences form a valuable information resource for determining their 2- and 3-dimensional structures as well as to identify further sequences of homology. These approaches are most easily facilitated by storing the sequences in a computer readable medium and then using the stored data in a known macromolecular structure program or to search a sequence database using well-known searching tools. Therefore, the invention further encompasses a computer readable medium (e.g. disks, tapes, chips, hard drives, compact disks, and video disks) having stored thereon one or more of the polynucleotide and/or polypeptide sequences of the invention (i.e. SEQ ID Nos: 1 to 41).

### EXAMPLES

As it will now be demonstrated by way of examples hereinafter, G1ORF67 is a very potent antibacterial compound binding selectively to S. aureus primary sigma factor. Although any methods and materials similar or equivalent to those described herein can be used in the practice or testing of the present invention, the preferred methods and materials are described.

### Example 1

### Identification of G1ORF67 as an Inhibitory ORF From Staphylococcus aureus Bacteriophage G1

Isolation and propagation of phages and preparation of phage genomic DNA followed published protocols (Sambrook et al., Molecular Cloning: A Laboratory Manual, (2001) Cold Spring Harbor Laboratory Press; Adams, M. H., Bacteriophages (Interscience Publishers, NY. 1959). The Staphylococcus aureus propagating strain PS15 [ATCC 23360], obtained from American Type Culture Collection (Manassas, Va., USA) was used as a host to propagate S. aureus bacteriophage G1 (“phage G1”). Phage G1 was isolated from a plaque by infecting the PS15 strain with a cocktail of Staphylococcus aureus bacteriophages (Bacteriophagum staphylococcum liquidum, lot number 361098) manufactured by BioPharm, Tbilisi, Republic of Georgia. The cocktail of Staphylococcus aureus phages was purchased from a drug store in Tbilisi. Phage G1 genomic DNA was randomly cleaved by sonication and the ends were repaired with T4 DNA polymerase and Klenow fragment and cloned into the Hincl site of plasmid pKS (Stratagene). The inserts were sequenced using BigDye™ primer or BigDye™ terminator cycle sequencing reactions (ABI Prism). Sequence contigs were assembled using Sequencher™ 3.1 (GeneCodes) or PhredPhrap/Consed™ 12.0 (CodonCode Corporation) software. The G1 phage genome was sequenced at least once in each direction. The assembled contig had at least three-fold coverage obtained from independent clones.

Bacteriophage ORFs encoded in phage G1 genome were identified as follows: beginning at the first nucleotide, the phage G 1 genome sequence is scanned for a start codon. When one is identified, the number of in-frame codons is counted until a termination codon is reached. A minimum threshold of 33 codons defines this bounded sequence as an ORF. This procedure is repeated, starting at the next nucleotide following the last stop codon, until the end of the phage sequence is reached. The scan is performed in an identical manner on all three reading frames of both DNA strands of the phage sequence, in order to identify all the putative ORFs. Putative genes are then identified based on the presence of a Shine-Dalgarno sequence within the 15 nucleotides upstream of the start codon. The E. coli-S. aureus shuttle vector pTOO21 (Tauriainen et al., Appl. Environ. Microbiol. (1997) 63:4456-64), containing the arsenite-inducible ars promoter and the arsR gene, was modified with an optimal Shine-Dalgarno sequence (AGGAGG) followed by a multiple cloning site (MCS). DNA encoding individual phage ORFs was amplified by PCR from phage genomic DNA and cloned into the MCS of the modified vector. Recombinant plasmids were introduced into S. aureus RN4220 by electroporation (Schenk and Laddaga, FEMS Microbiol. Lett. (1992) 73:133-138) and clones were selected on tryptic soy agar plates containing 30 μg/ml kanamycin (TSA/Kan). Phage ORFs that inhibited the growth of S. aureus were identified in a dot screen on TSA/Kan+/−5 μM NaAsO₂. Inhibitory ORFs were further characterized in growth kinetics assays as followed: Clones of S. aureus RN4220 harboring either the inhibitory ORF or a control noninhibitory ORF were grown in TSB/Kan+/−5 μM NaAsO₂. At different time intervals, aliquots of the cultures were plated onto TSA/Kan for determination of colony-forming units (CFU). Results are averages of three independent clones for each ORF+/−S.D.

Following the scheme described above phage G1ORF067 was identified as a bacterial growth inhibitory ORF. The bacterial growth inhibition kinetics for G1ORF067 is shown in FIG. 1. The number of CFU was significantly reduced in cells expressing G1ORF67. In contrast, when G1ORF67 is not expressed, the growth rate was similar to that observed with transformants harboring a non-inhibitory control ORF under both induced and non-induced conditions. The expression of G1ORF67 is bacteriostatic as it suppresses the logarithmic expansion of, the host culture such that the number of CFUs remains constant over time in induced cells compared to non-induced cells. When colony plating was done in the absence of kanamycin, the antibiotic necessary to maintain the selective pressure for the plasmid encoding ORF, the extent of growth inhibition was similar to plating in the presence of kanamycin. This confirms that the observed inhibitory effect was solely caused by overexpression of G1ORF67.

Nucleotide and amino acid sequences of G1ORF67 from phage G1 are set forth in SEQ ID NO: 6 and SEQ ID NO: 7 respectively. Both sequences were blasted against GenBank™ database, but no significant identity or homology was found (maximum identity being about 30%).

### Example 2

### Identification of a S. aureus Protein Target of Inhibitory ORF G1ORF67

To identify the S. aureus protein(s) that interacts with inhibitory ORF 67 of S. aureus bacteriophage G1, G1ORF67 was expressed and purified as a protein fusion from E. coli. The purified protein was cross linked to Affigel 10™ and incubated with cell lysate prepared from S. aureus. After extensive washes with affinity chromatography buffer containing increasing salt concentration, bound proteins were eluted with 1% SDS. The elution profile was assessed by SDS-polyacrylamide gel electrophoresis (SDS-PAGE) followed by silver staining.

Accordingly, a predominant band of a molecular mass of about 46 kDa, specifically retained by the G1ORF67 affinity column, was eluted with 1% SDS. The identity of the eluted protein was determined by mass spectrometry and the direct interaction of the identified protein with G1ORF67 was validated by a variety of in vitro and in vivo approaches as described below.

Cloning, Expression and Purification of G1ORF67 as a Recombinant Protein

Description of Vectors Used for Expression and Purification of Recombinant Proteins:

All the vectors used in this invention to generate recombinant clones for expression and purification from E. coli were derived either from pGEX-6P1™ (Amersham-Pharmacia Biotech) or pQE-80L™ (Qiagen). pQE-80L™ encodes an amino terminal 6× Histidine tag whereas pGEX-6P1™ encodes an amino terminal glutathione-S-terminal (GST) fusion followed by a PreScission™ protease recognition site enabling the recombinant protein to be cleaved from the GST portion after purification. To express the recombinant proteins, E. coli strain BL21 (Amersham-Pharmacia) was used as a host for all the recombinant clones. Oligonucleotides used to clone G1ORF67 and its deletion mutants (two hybrid analysis) are listed in SEQ ID NOs: 21 to 28.

Vector pGK was obtained by cloning synthetic annealed heart muscle kinase (HMK) oligonucleotide corresponding to the HMK phosphorylation site [Kaelin et al., 1992 Cell #70: 351-364], into pGEX-6P1™ linearized with BamHI-SalI. A similar procedure was used to generate the pGB, a double tagged expression vector encoding for GST as a fusion with the short version of the biotin acceptor domain annealed with its complementary strand [Beckett at al., 1999 Protein Science #8: 921-929] into pGEX-6P1™ linearized with BamHI-SalI.

The construction of pH6K, an expression vector encoding for an 6×amino terminal Histidine tag as a fusion with the HMK phosphorylation site was performed by inserting annealed HMK oligonucleotide into the BamHI and SalI sites of the pQE-80L™; the vector was renamed pH6K. A similar procedure was used to generate pHB, a double tagged expression vector encoding 6X-histidine tag and the short version of the biotin acceptor domain. The integrity of the sequences was confirmed by DNA sequencing.

G1ORF67 was sub-cloned from G1pTMORF67 into pHB and pGB vectors. Both vectors are E coli expression vectors used for in-frame N-terminal translational fusions with either as His-Bio double tag for pHB vector (6× Histidine residues fused to a biotin acceptor domain) or as a GST-Bio double tag for pGB vector (GST protein fused to a biotin acceptor domain). To this end, the G1 pTMORF67 plasmid was digested with BamHI and HindIII and the insert corresponding to G1ORF67 was cloned into the unique BamHI and HindIII of pHB and pGB vectors. The ligation product was used to transform E. coli strain BL21. Recombinant clones were obtained and the integrity of the sequence of G1ORF67 insert, in both vectors, was verified by DNA sequencing. The recombinant plasmids were referred to as G1 pHBORF67 (and the purified protein was referred to as HB-G1ORF67) or G1 pGBORF67 (and the purified protein was referred to as GB-G1ORF67).

Protein Expression and Purification:

To express HB-G1ORF67, 2 L of cells were grown in 2×YT broth media at 37° C. to an OD₆₀₀ of 0.4 to 0.6 (1 cm path length) and then induced with 0.4 mM isopropyl-1-thio-β-D-galactosidase (IPTG) for 3 h at 30° C. To express GB-G1ORF67, 6 L of cells were grown in 2×YT broth media at 37° C. to an OD₆₀₀ of 0.4-0.6 and then induced with 0.6 mM IPTG for 6 h at 25° C. After induction, cells were collected by centrifugation for 10 min at 7500 rpm using a Beckman JA-10™ rotor and used to purify the recombinant proteins.

To purify HB-G1ORF67, the bacterial pellet was resuspended in 40 ml of His buffer (20 mM Hepes pH 8.0, 500 mM NaCl and 10 mM Imidazole) supplemented with 0.1 mg/ml of lysozyme and one tablet of protease inhibitor cocktail (Roche Diagnostics). Cells were lysed for 15 min on ice followed by 5 bursts of sonication (1 min/burst) and then 1% of Triton X-100™ was added to the lysate. After 30 min of incubation with end-over-end rotation at 4° C., the lysate was centrifuged for 30 minutes at 19,000 rpm using a Beckman JA-20™ rotor. The supernatant was applied to a 2 ml bed volume of Nickel-NTA agarose (Qiagen) for 30-60 min with end-over-end rotation at 4° C. The flow-through was collected, the beads were washed with 30 ml His buffer and eluted with 200 mM imidazole in His buffer.

To purify GB-G1ORF67, the bacterial cell pellet was dissolved in 70 ml HNG-500 buffer (20 mM Hepes pH 8.0, 500 mM NaCl and 10% glycerol) supplemented with 0.1 mg/ml of lysozyme, 1 mM DTT (Gibco BRL), 1 mM PMSF (Sigma) and one tablet of protease inhibitor cocktail (Roche Diagnostics). Cells were lysed for 15 min on ice followed by 5 bursts of sonication (30 s/burst) and then, 1% of Triton X-100™ was added to the lysate. After 30 min of incubation with end-over-end rotation at 4° C., the lysate was centrifuged for 40 minutes at 19 000 rpm at 4° C. using a Beckman JA-20™ rotor. The supernatant was applied to a 7.5 ml bed volume of glutathione Sepharose™ (Sigma) and mixed for 30 min at 4° C. with end-over-end rotation. The flow-through was collected, the beads were washed with 20 ml HNG-150 buffer (20 mM Hepes pH 8.0, 150 mM NaCl and 10% glycerol), with 10 ml of HNG-1000 buffer (20 mM Hepes pH 8.0, 1 M NaCl and 10% glycerol) and with 50 ml of HNG-150 buffer (20 mM Hepes pH 8.0, 150 mM NaCl and 10% glycerol). Proteins were eluted with 10 mM reduced glutathione (Sigma) in HNG-150 buffer.

Eluted proteins were analyzed by a 10% SDS-PAGE and visualized by staining with Coomassie Brilliant Blue™ R250 stain to assess purity of purified HB-G1ORF67 and GB-G1ORF67. The protein concentration was determined with the BioRad™ kit using gamma globulin protein as a standard. Proteins were divided into aliquots and stored at −80° C.

Preparation of Cell Lysate from S. aureus RN4220 Strain:

The lysate was prepared from cell pellets of exponentially growing cells using lysostaphin digestion followed by sonication and nuclease digestion. The cell pellet (˜4.5 g) was suspended in 10 ml of lysis buffer (20 mM Hepes pH 7.5, 150 mM NaCl, 10% glycerol, 1 mM DTT, 1 mM PMSF, one tablet of protease inhibitor cocktail (Roche Diagnostics™), 20 μg/ml of each RNase A and DNase 1 and 1250 units of Lysostaphin (Sigma). The cell suspension was incubated at 37° C. for 1 h, cooled to 4° C., and made up to a final concentration of 1 mM EDTA and 500 mM NaCl. The lysate was sonicated on ice using 5 bursts of 20 seconds each. The lysate was made up to 1% Triton X-100™, mixed by end-over-end rotation for 30 minutes at 4° C. and centrifuged at 30 000 rpm for 3 hrs at 4° C. using a Beckman Ti70™ fixed angle rotor. The supernatant was collected and dialyzed overnight against affinity chromatography buffer (ACB; 20 mM Hepes pH 7.5, 10% glycerol, 1 mM DTT, and 1 mM EDTA) containing 100 mM NaCl, and 1 mM PMSF. The dialyzed lysate was centrifuged at 19 000 rpm in a Beckman JA-20™ rotor for 1 hr at 4° C. and the protein concentration of the supernatant was determined using the BioRad™ kit and gamma globulin protein as a standard. Samples were divided into aliquots and stored at −80° C.

Affinity Chromatography:

GST and HB-G1ORF67 were cross-linked to Affigel 10™ resin (BioRad) at protein/resin concentrations of 0 and 7 mg/ml. The cross-linked resin was then blocked with 10 mM ethanolamine for 1 h at 4° C., equilibrated with ACB buffer containing 100 mM NaCl and dispensed in microtubes as 40 μl bed volume aliquots. S. aureus lysate was centrifuged at 4° C. in a micro-centrifuge for 15 minutes and 400 μl of the supernatant were mixed with the matrix and allowed to bind for 20-30 min at 4° C. with constant agitation by end-over-end rotation. After binding, the beads were pelleted by a brief centrifugation at 2 000 rpm, the pellet was resuspended in ACB buffer and transferred to a Multiscreen-R5™ 96-wells plate (Millipore). The beads were sequentially washed, 400 μl per wash, with ACB buffer, ACB buffer supplemented with 1% Triton X-100™, ACB buffer supplemented with 250 mM NaCl and ACB buffer supplemented with 1 M NaCl. Bound proteins were eluted with 1% SDS, resolved on a 10% SDS-PAGE gel and visualized with the silver staining method. Proteins that were specifically retained by the HB-G1ORF67 affinity column were excised from the gel, fragmented with trypsin and the fragments were identified by mass spectrometry.

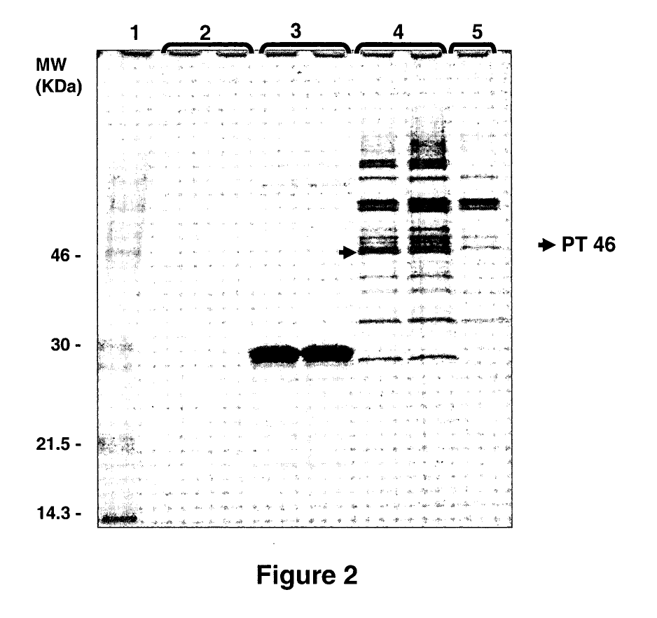

As shown in FIG. 2, a candidate polypeptide of ˜46 kDa (PT46; indicated by an arrow) was reproducibly recovered from affinity columns containing G1ORF67. PT46 was recovered primarily after elution with 1% SDS and was not observed in the GST control. Identification of S. aureus eluted proteins:

The candidate protein PT46 was excised from the gel and prepared for tryptic peptide digestion followed by mass determination by liquid chromatography (LC), electrospray tandem mass spectrometry (ms/ms) and the results were analyzed using the Mascot™ program (Matrix Science Inc.). The PT46 band was identified as an open reading frame (herein referred as ‘STAAU_R12’) corresponding the S. aureus RNA polymerase primary sigma factor (GenBank™ acc. No. 15927141).

Gen Essentiality Analysis of STAAU_R12:

The plasmids that were used for genetic modification of the STAAU_R12 locus were kindly provided by Dr. C. Y. Lee of University of Kansas Medical Center. The gene essentiality analysis of STAAU_R12 was carried out following the procedure described by Jana et al. [Plasmid (2000) 44:100-104] with the following modifications as follows. Specifically, a three-PCR fragment overlapping PCR was carried out and the product was cloned into pLL2443 [Jana et al., 2000 Plasmid #44,100-104] to construct a plasmid which was used to generate strain RpLLReR12 from S. aureus RN4220 in which the chromosomal STAAU_R12 gene is under the control of the spac promoter (a hybrid promoter of the E. coli lac operator and the B. subtilis SPOL phage promoter). Primers STAAU_R12-21 (5′-ccgctcgaggccatcaggcatggatccgg-3′; SEQ ID NO: 29) and STAAU_R12-22 (F3) (5′-gcgaggctagttacccttaagcttatctttaaatatgaacattcg-3′; SEQ ID NO: 30) were used to amplify a ˜1 kb fragment of RN4220 genomic DNA upstream the STAAU_R12 locus. Primers Cat-pSpac-F3 (5′-gataagcttaagggtaactagcctcgc-3′; SEQ ID NO: 31) and Cat-pSapc-R (5′-gaattcgatatcaagcttaattgttatccg-3′; SEQ ID NO: 32) were used to amplify the ˜2.3 kb Cat-T1₅-pSpac cassette from plasmid pLL2443. Primers STAAU_R12-23 (5′-cggataacaattaagcttgatatcgaattcatcggga ggccgtttcatg-3′; SEQ ID NO: 33) and STAAU_R12-24 (5′-aactgcagcttattcat gtcttggtatc-3′; SEQ ID NO: 34) were used to amplify a 1.2 kb fragment of the STAAU_R12 gene including its Shine-Dalgarno sequence and the initiation codon from RN4220 genomic DNA. An overlapping PCR with the above three PCR fragments was conducted and a ˜4.5 kb fragment was generated. This fragment was digested with XhoI and PstI and ligated into pLL2443 digested with XhoI and PstI. The resulting plasmid was used to transform strain RN4220. Transformants were grown at 42° C. to force integration of the plasmid into the genome by homologous recombination with chromosomal STAAU_R12 sequences. Resolution of the integrant generates a construct (RpLLReR12) in which STAAU_R12 is under the control of the spac promoter [Jana et al.,2000 Plasmid #44,100-104]. Construction of RpLLReR12 was validated by PCR and Southern analysis (data not shown). To regulate STAAU_R12 in RpLLReR12, plasmid pMJ8426 which expresses the E. coli lacI gene was introduced into RpLLReR12 to generate strain RpLLRMR12. The essentiality of STAAU_R12 was then evaluated by comparing bacterial growth of RpLLRMR12 in the presence and absence of 1.5 mM IPTG. Wild-type RN4220 was used as control. Growth of strain RpLLRMR12 on solid media was absolutely dependent on the presence of the inducer in the plate. This indicates that expression of STAAU_R12 is required for cell growth (data not shown).

To test the IPTG-dependency of RpLLRMR12 in growth kinetics, exponentially-growing RpLLRMR12 cells were diluted to an initial optical density at 565 nm (OD₅₆₅) of 0.05 and different amounts of IPTG (0-1.5 mM) were added. Growth kinetics were followed by measuring OD₅₆₅ every hour. The growth of strain RpLLRMR12 was IPTG-dependent, confirming essentiality of STAAU_R12 (data not shown).

### Example 3

### Validation of the Interaction Between G1ORF67 and STAAU_R12

The interaction between G1ORF67 and STAAU_R12, was validated using both cell-based (Yeast two hybrid system) and in vitro approaches (Far western and Time-Resolved Fluorescence Resonance Energy Transfer). Oligonucleotides used to clone STAAU_R12 and its deletion mutants (two hybrid analysis) are listed in SEQ ID NOs: 13 to 20.

Confirmation of the Interaction Between STAAU_R12 and G1ORF67 by the Yeast Two-Hybrid System Approach:

To validate the interaction between G1ORF67 and STAAU_R12, we first performed yeast two-hybrid analyses with full length and deletions within both STAAU_R12 and G1ORF67. The inserts were cloned into pGADT7™ and pGBK™, the yeast vectors for two hybrid system studies (Clontech Laboratories) as fusions with the Gal4 activation domain (for pGADT7™) as well as with the Gal4 DNA-binding domain (for pGBK™). pGADT7 and pGBK recombinant plasmids bearing different combinations of constructs were introduced into a yeast strain (AH109, Clontech Laboratories), previously engineered to contain chromosomally-integrated copies of E. coli lacZand the selectable HIS3 and ADE2 genes. Co-transformants were plated in parallel on a yeast synthetic medium (SD) supplemented with amino acid drop-out lacking tryptophan and leucine (TL minus) and on SD supplemented with amino acid drop-out lacking tryptophan, histidine, adenine and leucine (THAL minus). Under those conditions, growth on the selective SD THAL minus medium is strictly dependent on the interaction of STAAU_R12 with G1ORF67. Thus compounds or inhibitors of the interaction between G1ORF67 and STAAU_R12 can be evaluated for their direct consequence on cell viability.

Construction of Recombinant Vectors with the Full Length of STAAU_R12 and G1ORF67 for the Yeast Two-Hybrid System Analysis:

The full length nucleotide sequence of STAAU_R12 was PCR-amplified from genomic DNA of S. aureus strain RN4220 using sense (5′-cgGGATCCATGTCTGATAACACAGTT-3′; SEQ ID NO: 13) and antisense oligonucleotides (5′-acgcGTCGACTTAATCCATAAAGTCTTTC-3′; SEQ ID NO: 17) that include the predicted translation initiation and stop codons of STAAU_R12 gene (SEQ ID NO: 1). For convenient cloning, the sense and antisense oligonucleotides were flanked by BamHI and SalI respectively.

The PCR product was purified using the Qiagen™ purification kit and digested with BamHI and SalI. The digested PCR product gel was purified and cloned into the unique BamHI and SaA restriction sites of pGADT7™ and pGBK™ yielding thus pGADSTAAU_R12 and pGBKSTAAU_R12, respectively. G1ORF67 was cloned by digesting G1pTMORF67 with BamHI and HinDIII and the G1ORF67 insert was cloned into BamHI and HindIII restriction sites of pGADT7 and pGBK yielding thus G1pGADORF67 and G1pGBKORF67, respectively. This cloning thus allowed the yeast two-hybrid analysis to be done in both directions.

Construction of Recombinant Vectors with Deletion Fragments of STAAU_R12 and G1ORF67 for the Yeast Two-Hybrid System Analysis:

To delineate the minimal domain in both STAAU_R12 and G1ORF67, required for conferring the interaction between the inhibitory ORF and its target, six truncated fragments of the polypeptide sequence of STAAU_R12 (SEQ ID NO: 2) and six truncated fragments of the polypeptide G1ORF67 (SEQ ID NO: 7) were generated by PCR amplification either from S. aureus genomic DNA (for STAAU_R12 deletions) or from G1ORF67 insert (for G1ORF67 deletions). The PCR products were digested with BamHI and SalI for STAAU_R12 fragments or BamHI and HindIII for G1ORF67 fragments and ligated into pGADT7 and pGBK vectors. The oligonucleotides used for PCR amplification to generate deletion mutants of both STAAU_R12 and G1ORF67 are shown in SEQ ID NOs: 13 to 28.

Yeast Two-Hybrid Analysis:

The full length sequence of STAAU_R12 was cloned into pGADT7 and used to co-transform the yeast strain AH109 with pGBK containing the full length sequence of G1ORF67. As negative controls, LaminC and SV40 large T antigen were used in parallel. Co-transformants harboring the G1ORF67 polypeptide only grew on selective SD THAL minus medium in the presence of STAAU_R12 indicating thus, that STAAU_R12 interacts with G1ORF67 (data not shown). The interaction of STAAU_R12 with G1ORF67 is specific since co-transformants with appropriate control plasmids (pGBKT7LaminC or pGADT7-LargeT) were not viable on SD THAL minus medium (data not shown). The same data were obtained when G1ORF67 was cloned into pGADT7 and used with pGBKSTAAU_R12 to co-transform the yeast strain (not shown).

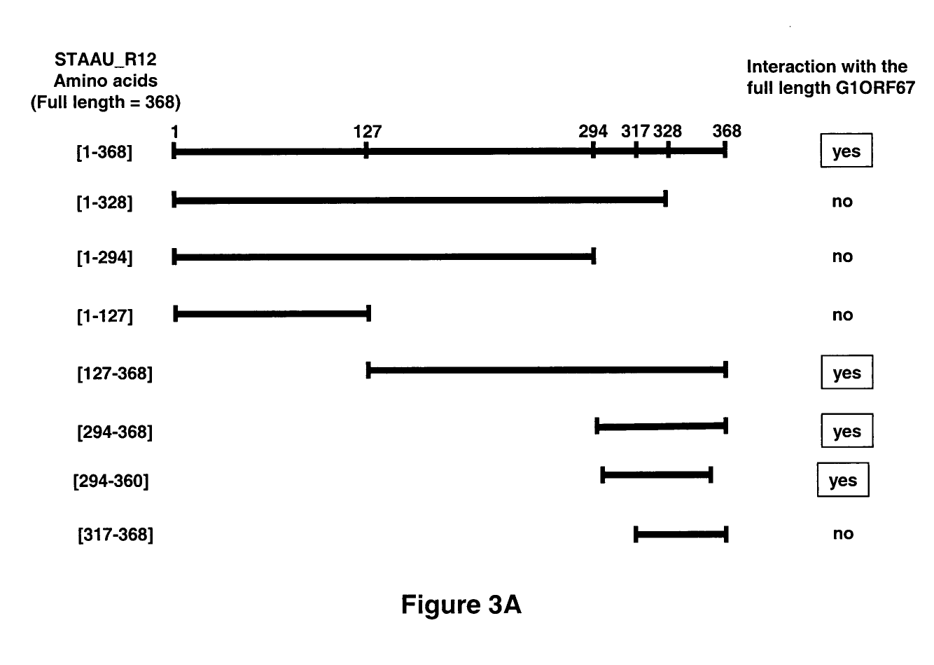

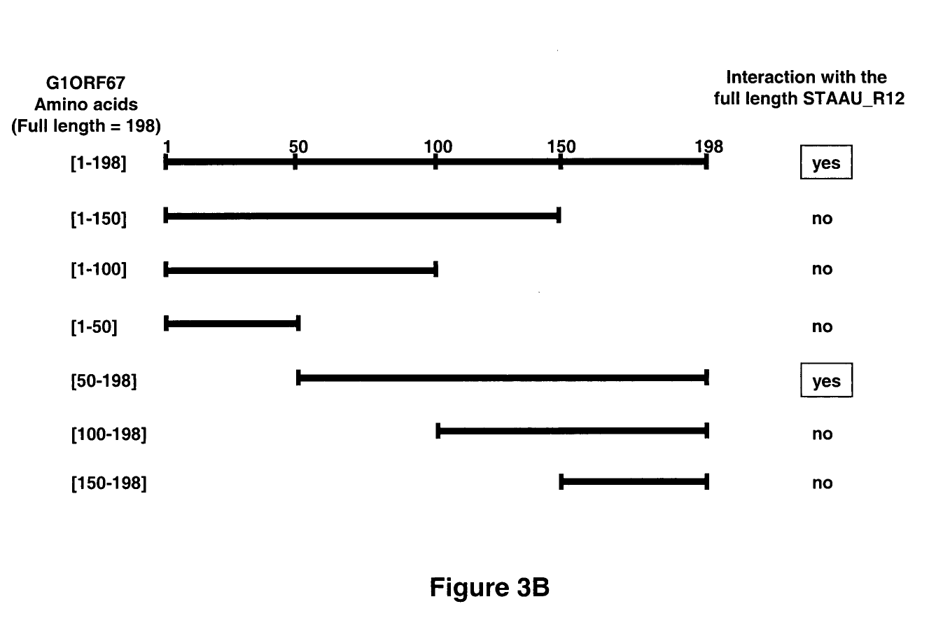

To define the minimal region of STAAU_R12 conferring an interaction with G1ORF67, six deletions of STAAU_R12 were cloned into pGADT7 and introduced into AH109 yeast cells with the pGBK vector containing the full length sequence of G1ORF67. The resulting co-transformants were analyzed for their ability to induce expression of reporter genes. FIG. 3A summarizes the results of interaction for each of STAAU_R12 truncated fragments with G1ORF67 polypeptide. Accordingly, the portion of STAAU_R12 extending from amino acids 294 to 360 (herein referred to as SEQ ID NO: 5 or STAAU_R12_{—}294_{—}360) was found to interact with G1ORF67 since the introduction of appropriate plasmids into host yeast cells resulted in their growth on THAL minus SD medium (in one direction, i.e. with R12_{—}294_{—}360 in activating domain and G1ORF67 in binding domain). This 67 amino acid sequence (SEQ ID NO: 5) represents the minimal region of STAAU_R12, identified by yeast two hybrid assay, that maintains the interaction ability with G1ORF67. This minimal region contains the domains 4.1 and 4.2 which have been described in E. coli to be required for conferring the interaction of sigma 70 and the T4 AsiA protein [Jeffrey et al., 2001 J. Biol. Chem. #276:41128-41132; Burgess and Anthony 2001 Cur. Opin, Microbiol. #4: 126-131]. Interestingly, another short but longer STAAU_R12 fragment (75 amino acids) corresponding to the portion of STAAU_R12 extending from amino acids residues 294 to 368 (herein referred to as SEQ ID NO: 4 or STAAU_R12_{—}294_{—}368) also interacted with G1ORF67, in both directions (i.e. R12_{—}294-368 in activating and in binding domain).

To define the minimal region of G1ORF67 conferring an interaction with STAAU_R12, six deletions of G1ORF67 were cloned into pGADT7 and introduced into AH109 yeast cells with the pGBK vector containing the full length sequence of STAAU_R12. The resulting co-transformants were analyzed for their ability to induce expression of reporter genes required for viability on selective medium. FIG. 3B summarizes the results of the interaction using truncated fragments of G1ORF67 with STAAU_R12 polypeptide. As shown, the portion of G1ORFF67 extending from amino acids residues 50 to 198 (herein referred to as SEQ ID NO: 8 or G1ORF67_{—}50_{—}198) was found to interact with STAAU_R12 since the introduction of appropriate plasmids into host yeast cells resulted in their growth on THAL minus SD medium (results not shown). This 149 amino acid sequence (SEQ ID NO: 8) represents the minimal region of G1ORF67, identified by yeast two hybrid assay, that maintains the interaction ability with STAAU_R12. The same results were obtained when fragments of G1ORF67 were cloned into pGBK and used to cotransform the yeast strain with pGBK containing the full length of STAAU_R12 (data not shown).

Confirmation of the Interaction Between STAAU_R12 and G1ORF67 In Vitro by Far Western and TR-FRET Assays:

To characterize the interaction between STAAU_R12 and the inhibitory ORF67 of S. aureus bacteriophage G1, the recombinant proteins were expressed in E. coli as Histidine and GST fusions using pHB, pH6K and pGB vectors. Purified proteins were used in Far western and TR-FRET.

Cloning, Expression and Purification of STAAU_R12 as Recombinant Protein from E. coli:

Construction of pGKSTAAU_R12 and pH6KSTAAU_R12 was initiated by digesting pGADSTAAU_R12 with EcoRI and XhoI restriction enzymes. The insert corresponding to STAAU_R12 was gel purified and ligated into the EcoRli and SalI restriction sites of pGK and pH6K. The integrity of the STAAU_R12 sequence was verified by DNA sequencing and the recombinant plasmids were used to transform E. coli strain BL21.

The overexpression and purification of STAAU_R12 used cells transformed with pH6KSTAAU_R12. To this end, 8 L of cells were grown in 2×YT broth media at 37° C. to an OD₆₀₀ of 0.4-0.6 and then induced with 1 mM IPTG for 2 to 3 hrs at 37° C. After induction, cells were collected by centrifugation for 10 min at 7 500 rpm using a Beckman JA-10™ rotor and used to purify the recombinant protein.

To purify STAAU_R12 as a Histidine tag fusion, cells were resuspended in approximately 120 ml of HNG-1000 buffer (20 mM Hepes pH 8.0, 1 M NaCl, 10% glycerol) supplemented with 10 mM imidazole and one tablet of protease inhibitor cocktail (Roche Diagnostics) and lysed by 5 bursts of sonication (1 min/burst). Triton X-100™ was added to the lysate to a final concentration of 1% of and mixed by end-over-end rotation for 30 min at 4° C. The lysate was centrifuged at 19 000 rpm using a Beckman JA-20™ rotor for 30 min at 4° C. The supernatant was applied to a 10 ml bed volume of Nickel-NTA agarose (Qiagen) and allowed to flow by gravity. The column was successively washed with 40 ml of HNG-1000 buffer (20 mM Hepes pH 7.5, 1 M NaCl and 10% glycerol) supplemented with 10 mM imidazole followed by a wash with 30 ml of HNG-150 buffer (20 mM Hepes pH 7.5, 150 mM NaCl and 10% glycerol) supplemented with 10 mM imidazole. Bound proteins were eluted with 200 mM imidazole in HNG-150 buffer (20 mM Hepes pH 7.5, 150 mM NaCl and 10% glycerol).

To monitor the degree of purity, the eluted protein was resolved by 12% SDS-PAGE and visualized by Coomassie staining. The degree of purity of STAAU_R12 was estimated to be approximately 60% and therefore, a gel filtration step was performed on the eluted protein. The protein was applied to a Superdex S₂₀₀™ gel filtration column (Amersham-Pharmacia) equilibrated in HNG-150 buffer (20 mM Hepes pH 7.5, 150 mM NaCl and 10% glycerol). Fractions containing STAAU_R12, as monitored by western blot analysis using an anti-His antibody (Sigma), were collected and pooled. Protein concentration was determined using the BioRad™ protein assay kit (BioRad) and the γ-globulin protein as a standard. The purified protein was divided into aliquots and stored at −80° C. and referred to as HK-STAAU_R12.

Far Western Analysis:

Radiolabeling of the proteins was done through the heart muscle phosphate acceptor site with the heart muscle kinase enzyme (HMK). The labeled probe was incubated with immobilized target protein, and the interaction was detected by exposure to X-ray film after extensive washes.

For radiolabeling with α-[³²P]-ATP, 10-15 μg of STAAU_R12 fused to 6× histidine and the HMK kinase acceptor domain (HK-STAAU_R12) were incubated with 50 units of the catalytic sub-unit of cAMP-dependent protein kinase (Heart Muscle Kinase; Sigma) in a total volume of 100 μl containing 20 mM Tris pH 7.5, 100 mM NaCl, 12 mM MgCl₂, 1 mM DTT and 50 μCi of [γ³²P]-ATP (3000 Ci/mmole) (NEN/Mandel) for 30 min at room temperature. To remove free nucleotides, the protein was applied to a Sephadex-G50™ column and eluted with Z-buffer (25 mM Hepes pH 7.7, 12.5 mM MgCl₂, 20% glycerol, 100 mM KCl & 1 mM DTT) and the incorporation of γ³²P-ATP was determined by counting in a liquid scintillation counter.

Increasing amounts (from 100 ng to 2 μg) of HB-G1ORF67 and as a negative control, 77ORF104 protein (2 μg) were used for Far-western analysis. Proteins were resolved by 15% SDS-PAGE and transferred onto a nitrocellulose membrane (Millipore). Immobilized proteins were subjected to a denaturation/renaturation with guanidine hydrochloride prior to addition of the labeled protein as follows: the membrane was treated with 6 M guanidine hydrochloride in HBB buffer (25 mM Hepes pH 7.7, 25 mM NaCl, 5 mM MgCl₂, 1 mM DTT) for 20 min at 4° C. The proteins were renatured in situ by a progressive dilution of guanidine hydrochloride in HBB buffer. The membrane was blocked for at least 1 h with 5% powdered milk in HBB supplemented with 0.05% NP-40 and for 45 min in 1% powdered milk in HBB supplemented with 0.05% NP-40.

The hybridization was performed overnight at 4° C. in 10 ml of hybridization buffer (20 mM Hepes pH 7.7, 75 mM KCl, 0.1 mM EDTA 2.5 mM MgCl₂, 0.05% NP-40 and 1% milk) containing ˜250,000 cpm/ml of α-[³²P]-ATP-labeled HK-STAAU_R12 protein as probe. The membrane was washed three times for 10 min with hybridization buffer and exposed to X-ray film.

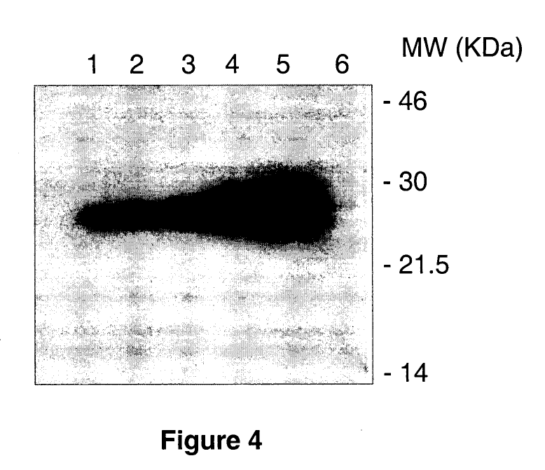

As shown in FIG. 4, a specific signal was observed when STAAU_R12 was used as labeled probe, against immobilized HB-G1ORF67 indicating that the inhibitory ORF G1ORF67 indeed directly interacts with STAAU_R12 in a dose-dependent manner. Using 77ORF104 protein as a negative control (see U.S. Pat. No. 6,376,652), the interaction between STAAU_R12 and G1ORF67 was shown to be specific. Confirmation of the interaction between STAAU_R12 and G1ORF67 by Time-Resolved Fluorescence Resonance Energy Transfer Assay “TR-FRET”:

TR-FRET technology was favorably used for monitoring the interaction of S. aureus phage G1ORF67 with its cognate target STAAU_R12 from S. aureus. This method is based on the energy transfer from a long-lived fluorophore (the energy donor) to anotherfluorophore (the energy acceptor) upon excitation of the donor (Mathis G., Clinical Chemistry (1995) 41:1391-1397). For this assay, Europium cryptate (Eu)-labeled anti-Histidine antibodies were used as energy donor and allophycocyanin (APC)-labeled anti-GST antibodies as energy acceptor. When the fluorescence donor and acceptor molecules are brought into close proximity as a result of a biomolecular interaction, flash excitation of Eu at 340 nm results in transfer of a portion of excitation energy non-radiatively to the APC acceptor which emits light at 665 nm. Under those conditions, the emission of light from APC adopts the time-resolved nature of the donor. Under conditions of no interaction and therefore of no proximity between the proteins under investigation, only background levels of emission from APC at 665 nm are detected.

To demonstrate the interaction between the inhibitory ORF and its target, G1ORF67 polypeptide was expressed and purified as a GST-Bio fusion (GB-G1ORF67) whereas STAAU_R12 polypeptide was expressed as a 6× Histidine fusion (HK-STAAU_R12). Thus under the TR-FRET conditions, the interaction of HK-STAAU_R2 with GB-G1ORF67 induces a transfer of energy from Eu to APC, detected optimally at 665 nm. In this assay, the addition of an inhibitor of the interaction between STAAU_R12 and G1ORF67 will result in a decrease in energy transfer from the donor to the acceptor.

To study the interaction between G1ORF67 and STMU_R12 by TR-FRET, increasing amounts (0 to 256 nM of GB-G1ORF67) were incubated with increasing amounts (0 to 256 nM) of HK-STMU_R12 in a volume of 24μ containing 20 mM Tris pH 8.0, 1 mM EDTA, 0.01% Triton X-100™, 400 mM potassium fluoride and 200 nM BSA. The reaction was incubated for 1 h at room temperature and then, 6 μl of a mixture of Eu conjugated anti-Histidine (CIS International, USA) and APC conjugated anti-GST (Prozyme) were added to final concentration of 3 and 15 nM respectively. Samples were mixed and 25 μl of the mixture was transferred to a 96-well opaque plate (Molecular Devices). After 45 min incubation at room temperature, the fluorescence emission at 665 nm (APC) and at 612 nm (EU) arising from excitation at 340 nm was measured using a Tecan Ultra™ plate reader.

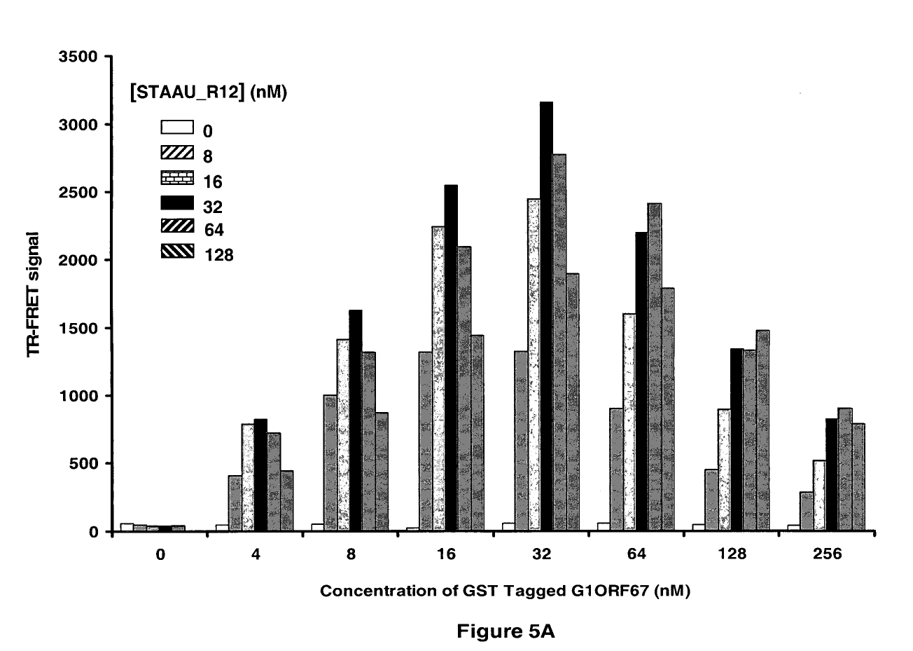

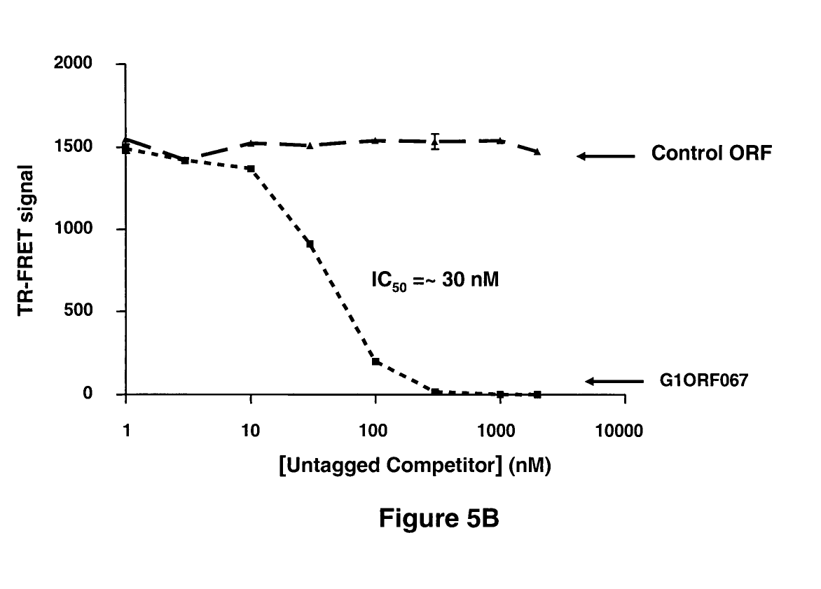

As shown in FIG. 5A, incubation of the appropriate amount of G1ORF67 and STAAU_R12 resulted in a high TR-FRET signal indicating that G1ORF67 and STAAU_R12 interact with each other. The assay was performed in duplicated with consistent results; the results of a single experiment are shown. Given that 32 nM of both G1ORF67 and STAAU_R12 were appropriate to obtain a high TR-FRET signal-to-background (30:1 ratio), we therefore used these concentrations to perform the IC₅₀ studies.

IC₅₀ studies were done exactly as above except that the reaction mixture contained increasing amounts of untagged G1ORF67 (the GST portion of the GB-G1ORF67 fusion protein was removed by cleaving the purified protein with PreScission™ protease). As a negative control, we used increasing amounts of untagged 77ORF104 (the GST portion as described). The assay was performed in duplicate.

As shown in FIG. 5B, increasing the amount of untagged G1ORF67 resulted in a sigmoidal inhibition curve indicating that the untagged G1ORF67 competes with the GST-tagged G1ORF67 for binding to STAAU_R12. In contrast, 77ORF104 which was used as a negative control did not show any inhibition. Under our experimental conditions, the concentration of competitor required to reach 50% inhibition (IC₅₀) was evaluated to about 30 nM.

### Example 4

### Effect of G1ORF67 on the Activity of STAAU_R12

STAAU_R12 is predicted to play a key role in directing the core polymerase enzyme for efficient transcription. As shown above by different methods, the interaction between G1ORF67 and STAAU_R12 was validated by both cell-based and in vitroapproaches. We therefore investigated the functional consequence of such interaction on the activity of STAAU_R12. To this end, we performed a series of in vitro experiments (in vitro transcription assays, DNA binding studies) as well as cell-based studies (macromolecular synthesis studies). In these experiments, we showed a marked and specific inhibition of STAAU_R12 activity by G1ORF67.

In Vitro Transcription Studies:

In vitro transcription studies were performed either by loading the radiolabeled RNA visualization product on polyacrylamide/urea gels followed by autoradiography to visualized the product or by performing a trichloroacetic acid (TCA) precipitation of the nascent radiolabeled transcript followed by scintillation counting.

In Vitro Transcription Assay and RNA Analysis:

Transcription reactions were performed with reconstituted holoenzyme from purified HK-STAAU_R12 and the E. coli core enzyme (Epicentre). The E. coli holoenzyme (Epicentre) was used at 10 nM as a positive control for monitoring transcription efficiency. Assays were done with increasing amounts (0 to 500 nM) of STAAU_R12 and 25 nM of E. coli core in a total volume of 25 μl containing 40 mM Tris-acetate pH 7.9, 100 mM NaCl, 5 mM MgCl₂, 1 mM DTT, 0.1 mg/ml BSA, 0.5 mM of each ATP, GTP, CTP, 0.25 mM UTP, 5 μCi [³²P] UTP (3000 Ci/mmole), 2 units of RNasin (Promega) and 40 ng of pB6 template DNA (Lutz and Bujard, Nuc. Acid Res. (1997) 6:1203-10) harboring λPL promoter, kanamycin gene and the Col E1 RNA-1. To investigate the in vitro effect of G1ORF67 on STAAU_R12-mediated transcriptional activation, G1ORF67 and other negative controls (0 to 2 μM) were pre-incubated with STAAU_R12 for 10 min on ice prior to addition of the other reagents. Reactions were incubated at 37° C. for 15 min and stopped by addition of 5 μl of loading buffer (95% formamide, 20 mM EDTA and 0.05% of each xylene cyanol and bromophenol blue). Samples were boiled for 5 min and electrophoresed on a 6% polyacrylamide/8 M urea gel.

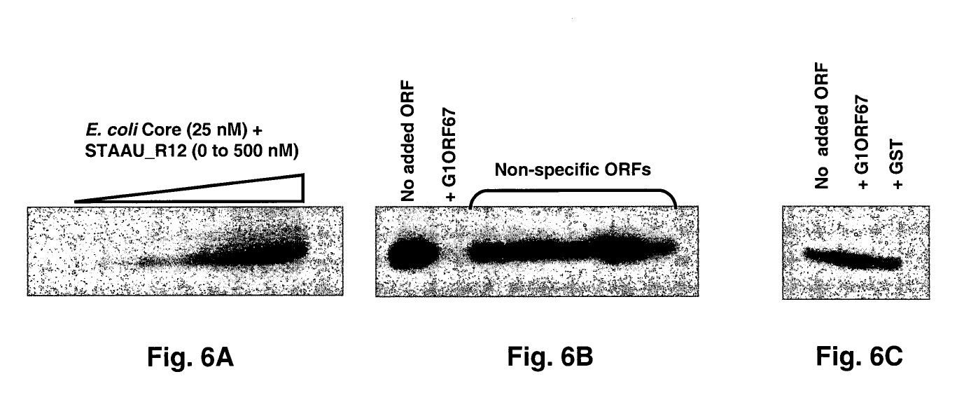

As shown in FIG. 6A, purified STAAU_R12 stimulated, in a dose-dependent manner transcription by the E. coli core polymerase from pB6 template DNA. The stimulation observed with this reconstituted heterogeneous holoenzyme is comparable to that conferred by the homogenous holoenzyme from E. coli (FIG. 6C). Our results are in a perfect agreement with results reported by other groups showing a STAAU_R12-dependent stimulation of transcription [Rao et al., 1995 J. Bacteriology 1995 #177: 2609-2614; Deora and Misra 1996 The Journal of Biological Chemistry #271: 21828-21834). The effect of G1ORF67 on the activity of STAAU_R12 was next functionally validated using this in vitrotranscription assay. As shown in FIG. 6B, pre-incubation of STAAU_R12 with G1ORF67 resulted in a dramatic inhibition of STAAU_R12-mediated transcriptional activation. This effect was specific to G1ORF67 since the other ORFs used in this study as negative controls did not show any inhibition of transcription. The observed effect of G1ORF67 was likely more specific for STAAU_R2 since pre-incubation of G1ORF67 with the E. coli holoenzyme did not affect transcription (FIG. 6C).

In Vitro Transcription Assay Using the Core RNA Polymerase From S. aureus and TCA Precipitation Assay

In order to convert this gel-based transcription assay to a 96-well high through put format, we used an in vitro transcription assay followed by a TCA precipitation step in order to separate radiolabeled nucleotides incorporated into nucleic acid from unincorporated label. Such format is convenient as it will allow rapid screening with a large collection of compounds. In this TCA precipitation assay, we reconstituted the holoenzyme from STAAU_R12 and S. aureus core enzyme instead of using the E. coli core enzyme with STAAU_R12.

Purification of S. aureus Core Enzyme:

In order to purify the core enzyme from S. aureus, we took advantage of the tight interaction between the different subunits of the core RNA polymerase. To this end, the S. aureus rpoA gene (encoding the α sub-unit) was PCR-amplified from genomic DNA of S. aureus strain RN4220 using the sense oligonucleotide (5′-cgGGATCCATGATAGAAATCGAAAAACCTAGA-3′; SEQ ID NO: 35) and the antisense primer (5′-acgcGTCGACACTATCTTCTTTTCTTAATCCTAA-3′; SEQ ID NO: 36). For convenient cloning, BamHI and SalI were added to the 5′ flanking region. The PCR product was cloned into the E. coli-S. aureus shuttle vector as a 6× Histidine/Biotin acceptor domain fusion under the control of ars-induced promoter. The integrity of the rpoA sequence was verified by DNA sequencing and the vector was referred to as pTMHB rpoA and used to transform S. aureus strain RN4220.

Transformed cells (30 L) were grown in TSB with kanamycin until an OD₅₄₀ of 0.5 was reached and then induced with 10 μM of sodium arsenite for 2 h at 37° C. and collected by centrifugation at 7 500 rpm for 10 min using a Beckman JA10™ rotor. The bacterial pellet was resuspended in 400 ml HNG-1 000 buffer (20 mM Hepes pH 8.0, 1 M NaCl and 10% glycerol) supplemented with 10 mM imidazole, one tablet of protease inhibitor cocktail (Roche Diagnostics), 1 mM PMSF (Sigma) and approximately 30 000 units of lysostaphin (Sigma). The cell suspension was incubated at 37° C. for 30 min with constant agitation followed by 3 bursts of sonication (30 s/burst). The cell debris was removed by centrifugation for 40 min at 19 000 rpm at 4° C. using a Beckman JA-20™ rotor.

Nucleic acids were precipitated with 3% of streptomycin sulfate (Sigma) for 20 min at 4° C. and centrifuged for 20 min at 19 000 rpm using a Beckman JA-20™ rotor. The supernatant “S1” was kept on ice and the pellet consisting of nucleic acids was extracted with 50 ml HNG-1 000 for 10 min at 4° C. with agitation and then centrifuged for 20 min at 19 000 rpm at 4° C. in a Beckman JA-20™ rotor. The supernatant “S2” was collected and pooled with S1 supernatant and applied to a 15 ml Nickel-NTA resin column (Qiagen). The flow through was collected and the beads were successively washed with 100 ml HNG-150 buffer (20 mM Hepes pH 8.0, 150 mM NaCl and 10% Glycerol) supplemented with 10 mM imidazole, 50 ml HNG-1 000 buffer supplemented with 10 mM imidazole, 25 ml HNG-150 supplemented with 10 mM imidazole and finally with 25 ml of TGEN (10 mM Tris pH 8.0, 5% glycerol, 150 mM NaCl and 0.1 mM EDTA) supplemented with 20 mM imidazole.

Proteins were eluted with 200 mM imidazole in TGEN buffer, analyzed by SDS-PAGE and the protein concentration was determined with the BioRad™ kit using the gamma globulin protein as a standard. Proteins were divided into aliquots and stored at −85° C. The identity of the purified subunits was confirmed by mass spectrometry (data not shown).

In Vitro Transcription and TCA Precipitation:

Transcription reactions were performed with reconstituted holoenzyme from purified HK-STAAU_R12 and purified S. aureus core enzyme. The E. coli core enzyme (Epicentre) was used as a positive control for monitoring transcription efficiency. The set up of high throughput screening assay for STAAU_R12 is illustrated in FIG. 7. Assays were done with 100 nM of STAAU_R12 and 50 nM of S. aureus core in a total volume of 25 μl containing 40 mM Tris-acetate pH 7.9, 100 mM NaCl, 5 mM MgCl₂, 1 mM DTT, 0.1 mg/ml BSA, 150 μM of each ATP, GTP, CTP, 30 μM UTP, 100.000 cpm of α-[³²P] UTP (3000 Ci/mmole), 1 U of RNasin (Promega) and 40 ng of pTMSM template DNA (a pTM derivative vector). To investigate the in vitroeffectofG1ORF67 on STAAU_R12 mediated transcriptional activation, G1ORF67 and a negative control (10 μM) were added to the mixture and the reaction was incubated for 1 h at 37° C. in a 96-well PCR-plate (BD-Falcon™). Samples were transferred to a 96-well Multiscreen™ plate (Millipore) and subjected to a 10% TCA precipitation step for 1 h at 4° C. in the presence of 10 μg of ss DNA as a carrier in a total volume of 200 μl. After filtration, the wells were washed 3 times (225 μl per wash) with 10% TCA, 2 times (225 μl per wash) with 95% ethanol and then 75 μl of liquid scintillation cocktail was added and the plates were counted for 30 s on a Trilux™ Microbeta counter (Perkin Elmer-Packard).

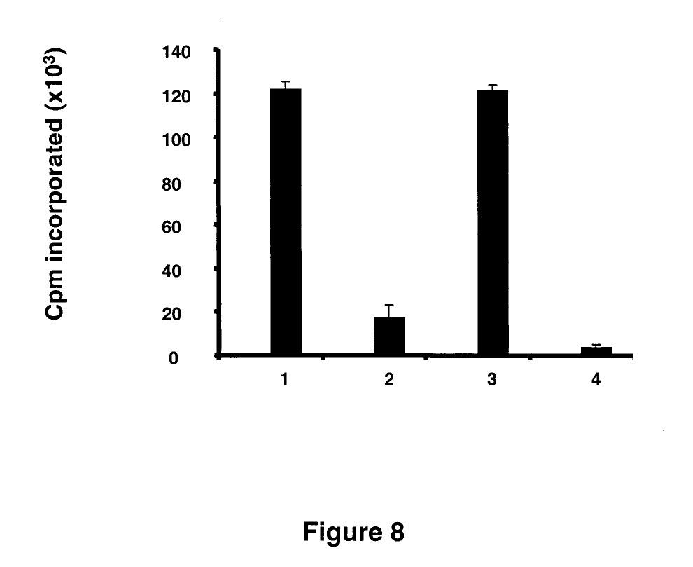

In the absence of STAAU_R12, the core enzyme had a very weak transcriptional activity (not shown), however, when STAAU_R12 was added to the core, transcription from template DNA is stimulated 5-10 fold (FIG. 8: Bar 1). Again, the effect of G1ORF67 on the activity of STAAU_R12-mediated transcriptional activation was functionally validated in this in vitro transcription assay. As shown also in FIG. 8, addition of G1ORF67 to the reaction mixture showed a dramatic inhibition of STAAU_R12 mediated transcriptional activation (Bar 2). This effect was specific to G1ORF67 since GST protein used in parallel as a negative control did not show any inhibition of transcription (Bar 3). The product made under these conditions corresponds indeed to RNA as judged from RNase A digestion (Bar 4).

In Vitro DNA-Binding Studies:

To complement our functional studies showing G1ORF67 inhibition of STAAU_R12-mediated transcription, we investigated the effect of G1ORF67 on the DNA-binding property of STAAU_R12 to an appropriate promoter. To this end, we performed a series of electrophoretic mobility shift assays, as a well as a TR-FRET assay.

Electrophoretic Mobility Shift Assay for Monitoring DNA-Protein Interaction:

Gel shift assays using a truncated lambda PR (λP_{R}) promoter from −41 to −12 (ATGATATTGACTTATTGAATAAAATTGGGT (SEQ ID NO: 37) annealed to ACCAATTTTATTCAATAAGTCAATATCAT (SEQ ID NO: 38)) as an oligonucleotide probe were performed essentially as described previously [Fenton et al. 2000 EMBO J., 19: 1130-1137]. Briefly, increasing amounts (0 to 500 nM) of purified STAAU_R12 were incubated with the E. coli core polymerase (85 nM) for 10 min on ice in a final volume of 20 μl containing 20 mM Tris-HCl pH 8,100 mM KCl, 1 mM EDTA, 1 mM DTT, 0.1 mg/ml bovine serum albumin, 6 ng/ml poly dl-dC (Amersham-Pharmacia), 3.25% glycerol and 1 nM annealed DNA probe. The mixture was incubated for 20 min on ice and then resolved onto a 4% polyacrylamide native gel using 1×TBE at 140 V for approximately 2 h. The specificity of the interaction was monitored by including in the reaction mixture an excess (100×) of unlabeled parental λP_{R} double strand oligonucleotide as a specific competitor or unrelated HMK double strand oligonucleotide (Seq. above) as a non competitor. The effect of G1ORF67 on the DNA binding activity of STMU_R12 was performed by pre-incubating STAAU_R2 with G1ORF67 or other ORFs as negative control prior to addition of the other reagents. The effect was evaluated by comparing the effect of G1ORF67 on DNA-protein complex formation.

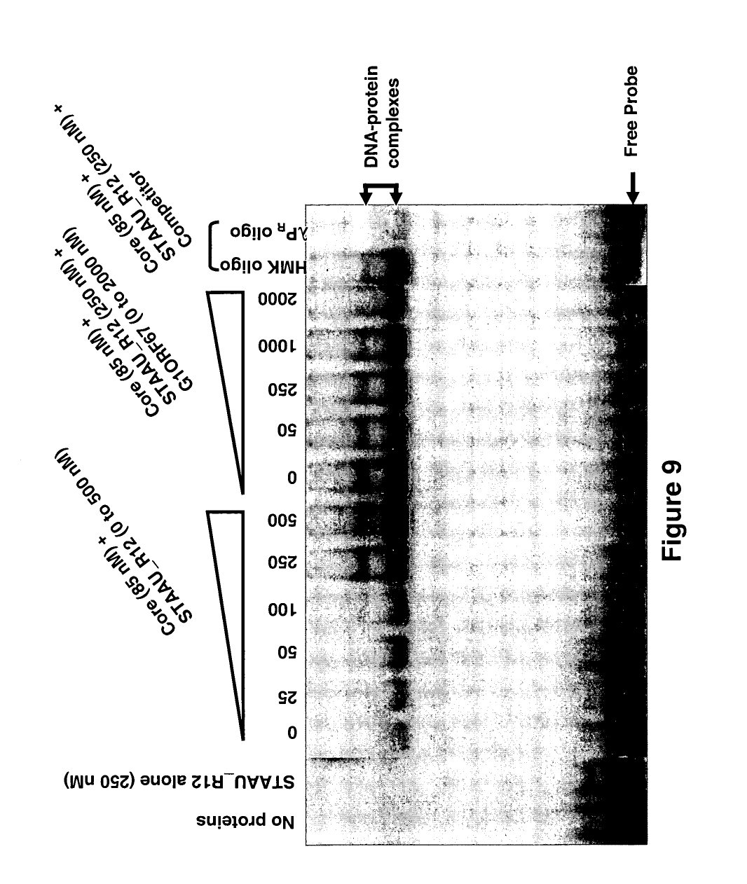

As shown in FIG. 9, in the absence of the core polymerase, STAAU_R12 is incompetent for binding to DNA; however when the E. coli core polymerase is added to the mixture, a DNA protein complex is formed an it is dose-dependent with respect to STAAU_R12. Using specific (parental λP_{R} oligonucleotide) and non specific (HMK oligonucleotide) competitors, the DNA-protein complex was shown to be specific since it can be only competed by the parental λP_{R} oligonucleotide (FIG. 9). Like the situation with E. coli σ⁷⁰, our data on STAAU_R12 are consistent with the fact that the core polymerase triggers a conformational change within sigma factor and thus convert it from an inactive to an active form [Dombroski et al., 1992 Cell #70: 501-512]. The ability of G1ORF67 to interfere with the DNA protein complex formation is shown in FIG. 9 suggesting that G1ORF67 acts by preventing STAAU_R12 from binding to DNA.

Time Resolved Fluorescence Resonance Energy Transfer “TR-FRET” assay for monitoring DNA-protein interaction:

We developed a fluorescence-based high throughput assay to monitor the interaction of STAAU_R12 with its cognate DNA. As described above, this method is based on the energy transfer from a fluorophore (the energy donor) to another fluorophore (the energy acceptor). Europium cryptate (Eu) labeled anti-Histidine was used as an energy donor and Allophycocyanin (APC) labeled streptavidin was used as an energy acceptor.

For this purpose, the 5′-end of the sense strand of the λP_{R} oligonucleotide (5′-ATGTTGACTTAAAGAATAAAATTGGGT-3′; SEQ ID NO: 39) was biotinylated and annealed to its complementary strand (5′-ACCCAATTTTATTCAATAAG TCAATATCAT-3′; SEQ ID NO: 38) and used as probe to study DNA-protein interaction with HK-STAAU_R12 (STAAU_R12 protein fused to 6× Histidine residues). After binding of HK-STAAU_R12 to its cognate DNA, both Eu donor and APC acceptor were added and the TR-FRET signal was measured as above. In this assay, the addition of an inhibitor of the interaction between STAAU_R12 and DNA such as a compound, untagged competitor like DNA or protein will result in an inhibition or a decrease of energy transfer from the donor to the acceptor.

To study the interaction between DNA and STAAU_R12 by TR-FRET, increasing amounts (0 to 256 nM of annealed oligonucleotide) were incubated with increasing amounts (0 to 128 nM) of HK-STAAU_R12 in a volume of 24 μl containing 10 nM E. coli core (Epicentre), 20 mM Hepes pH 8.0, 100 mM KCl, 1 mM EDTA, 400 mM potassium fluoride, 200 nM BSA and 3% glycerol. The reaction was incubated for 15 min at room temperature and then, 6 μl of a mixture of Eu-conjugated anti-Histidine (CIS International) and APC-conjugated streptavidin (Prozyme) were added to final concentrations of 3 and 15 nM, respectively. Samples were mixed and 25 μl of the mixture was transferred to a 96-well opaque plate (Molecular Devices). After 45 min incubation at room temperature, the emission at 665 nm (APC) and at 612 nm (Eu) arising from excitation as 340 nm was measured using a Tecan Ultra™ plate reader. To demonstrate the specificity of the interaction between STAAU_R12 and the λP_{R} oligonucleotide, a 0- to 5-fold molar excess of a specific competitor consisting of an untagged λP_{R} oligonucleotide (5′-ATGTTGACTTAAAGA ATAAAATTGGGT-3′ (SEQ ID NO: 39) annealed to 5′-ACCCAATTTTATTCAATAAGTCAATATCAT-3′ (SEQ ID NO: 38)) or untagged λP_{L} oligonucleotide (5′-GATAGAGTTGACATCCCTATCAGTGATAGAGATACTGAGAAC ATCAGC-3′ (SEQ ID NO: 40) annealed to 5′-GCTGATGTGCTCAGTATCTCTAT CACTGATAGGGATGTCMTCTCTATC-3′ (SEQ ID NO: 41)) was used. A mutated version of untagged λP_{R} oligonucleotide consisting of a substitution of the −35 region (from TTGACT in the wild type P_{R} oligonucleotide to ACTTTG in the mutated oligonucleotide) was used as a non specific competitor. Competitors were added to the reaction mixture prior to addition of Eu and APC conjugates. In order to investigate the effect of G1ORF67 on the DNA binding activity of STAAU_R12, increasing amounts (O to 4 μM) of G1ORF67 protein or 77ORF104 protein (negative control) were added to the reaction mixture prior to addition of Eu and APC conjugates. Experiments were performed in duplicate with consistent results; the results of a single experiment are shown.

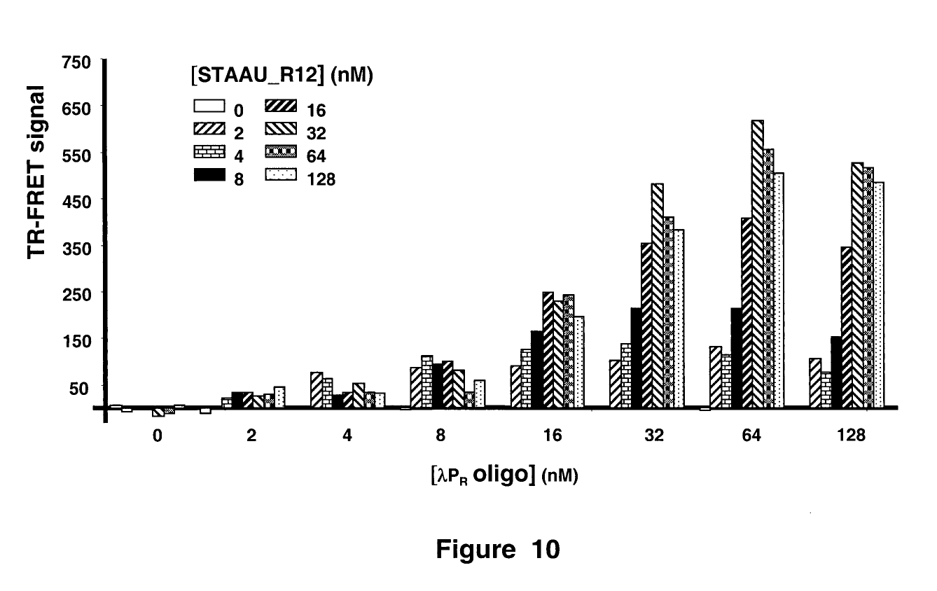

As shown in FIG. 10, incubation of the appropriate amount of both STAAU_R12 and DNA resulted in a significant TR-FRET signal indicating that STAAU_R12 forms a complex with the biotinylated promoter fragment DNA. For competition studies, we used 32 nM of HK-STAAU_R12 and 45 nM of biotinylated DNA. The formation of the DNA-protein complex was significantly reduced under the conditions where an excess of the parental λP_{R} oligonucleotide or the λP_{L} oligonucleotide was added to the mixture (data not shown). This observation, which is consistent with the fact that both oligonucleotides have a similar −35 binding site, is supported by competition studies with the mutated version of λP_{R} which failed to inhibit the binding of STAAU_R12 to the probe.

The ability of G1ORF67 to inhibit the core-dependent DNA binding property of STAAU_R12 was also tested. Increasing the amount of G1ORF67 (0 to 4 μM) showed a marked inhibition of the TR-FRET signal (7-fold reduction). In contrast, 77ORF104 which was used as a negative control did not show any inhibition when tested over the same range of concentrations (data not shown).

### Example 5

### Effect of G1ORF67 on the Synthesis of Macromolecules in a Cell-Based Assay

To complement the inhibitory effect of G1ORF67 observed in vitro on the activity of STAAU_R12, we investigated the effect of G1ORF67 on the synthesis of macromolecules in S. aureus. To this end, the effect G1ORF67 on STMU_R12 was evaluated in the S. aureus strain RN4220 expressing G1ORF67 under the control of the arsenite promoter, by measuring the uridine uptake to monitor global transcription. As negative controls, 77ORF104 and other phage ORFs were used in parallel. Briefly, exponentially growing cells harboring G1ORF67 or the negative control ORFs were induced or not with 5 μM of sodium arsenite for different periods of time and then, 100 μl of the cells were withdrawn and subjected to a pulse labeling with 0.1 μCi/ml of ³H-uridine (Amersham-Pharmacia) for 15 min at 37° C. Cells were then treated with 10% TCA for 30 min at 4° C. and unincorporated radioactivity was removed by filtration using Multiscreen™ 96-well plates (Millipore). The filters were washed with 10% TCA and 95% ethanol and then, liquid scintillation cocktail was added and the filters were counted using a Microbeta counter (Wallac).

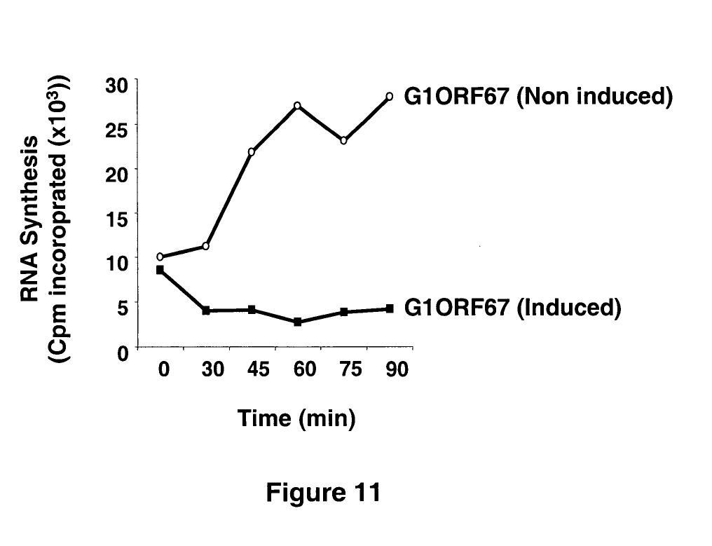

As shown in FIG. 11, expression of G1ORF67 resulted in a marked reduction of RNA synthesis as monitored by uridine uptake. In non induced cells, the effect of G1ORF67 is comparable to that observed in the negative control ORFs, whether induced or not (not shown). We also examined the effect of G1ORF67 on the synthesis of other macromolecules such as DNA and protein. Under our experimental conditions, in contrast to RNA synthesis, no inhibitory effect on DNA and protein synthesis was observed with G1ORF67 under induced conditions (data not shown), confirming the specificity of the inhibition in cells.

Although the present invention has been described hereinabove by way of preferred embodiments thereof, it can be modified without departing from the spirit and nature of the subject invention as defined in the appended claims.

What is claimed is:

1. A method for inhibiting bacterial growth, comprising contacting a bacterium with an antibacterial compound that specifically binds to a bacteriophage polypeptide binding domain of Staphylococcus aureus primary sigma factor polypeptide, wherein said bacteriophage polypeptide binding domain comprises an amino acid sequence selected from the group consisting of:
(i) the amino acid sequence set forth in SEQ ID NO: 3;
(ii) the amino acid sequence set forth in SEQ ID NO: 4;
(iii) the amino acid sequence set forth in SEQ ID NO: 5;
(iv) a fragment of (i) or (ii) containing said bacteriophage polypeptide binding domain; and
(v) a variant having at least 95% sequence identity with one of (i), (ii), (iii), and (iv), and containing a domain that is bound by said bacteriophage polypeptide.

2. The method according to claim 1, wherein said compound is a polypeptide selected from the group consisting of the polypeptide set forth in SEQ ID NO: 7, the polypeptide set forth in SEQ ID NO: 8, a fragment of the polypeptide set forth in SEQ ID NO: 7 or SEQ ID NO: 8, and a variant having at least 95% sequence identity with the polypeptide set forth in SEQ ID NO: 7 or SEQ ID NO: 8, wherein said fragment and said variant specifically bind to said bacteriophage polypeptide binding domain of said Staphylococcus aureus primary sigma factor polypeptide.

3. A method for inhibiting growth of Staphylococcus aureus, comprising contacting a Staphylococcus aureus bacterium with an antibacterial compound that specifically binds to a bacteriophage polypeptide binding domain of Staphylococcus aureus primary sigma factor polypeptide, wherein said bacteriophage polypeptide binding domain comprises an amino acid sequence selected from the group consisting of:
(i) the amino acid sequence set forth in SEQ ID NO: 3;
(ii) the amino acid sequence set forth in SEQ ID NO: 4; and
(iii) the amino acid sequence set forth in SEQ ID NO: 5.

4. An isolated or purified fragment of the S. aureus primary sigma factor polypeptide set forth in SEQ ID NO: 2, wherein said fragment comprises a bacteriophage polypeptide binding domain.

5. The fragment of claim 4, wherein said bacteriophage polypeptide binding domain comprises from about 67 to about 245 amino acids.

6. The fragment claim 4, wherein said a bacteriophage polypeptide binding domain is bound by the bacteriophage polypeptide set forth in SEQ ID NO:7 and/or the bacteriophage polypeptide set forth in SEQ ID NO:8.

7. The fragment of claim 4, wherein said bacteriophage polypeptide binding domain comprises an amino acid sequence selected from the group consisting of:
(i) the amino acid sequence set forth in SEQ ID NO: 3;
(ii) the amino acid sequence set forth in SEQ ID NO: 4; and
(iii) the amino acid sequence set forth in SEQ ID NO: 5.

8. An isolated or purified fragment of the S. aureus primary sigma factor polypeptide set forth in SEQ ID NO: 2, wherein said fragment comprises a bacteriophage polypeptide binding domain comprising an amino acid sequence selected from the group consisting of:
(i) the amino acid sequence set forth in SEQ ID NO: 3;
(ii) the amino acid sequence set forth in SEQ ID NO: 4;
(iii) the amino acid sequence set forth in SEQ ID NO: 5;
(iv) a fragment having at least 50 contiguous amino acids of (i) or (ii), and containing said bacteriophage polypeptide binding domain; and
(v) a variant having at least 95% sequence identity with one of (i), (ii), (iii), and (iv), and containing a domain that is bound by said bacteriophage polypeptide.

9. An isolated or purified bacteriophage polypeptide that binds the Staphylococcus aureus primary sigma factor polypeptide set forth in SEQ ID NO: 2.

10. An isolated or purified polypeptide comprising at least 10 contiguous amino acids of the amino acid sequence set forth in SEQ ID NO: 7, wherein said polypeptide binds the Staphylococcus aureus primary sigma factor polypeptide set forth in SEQ ID NO: 2, and/or wherein said polypeptide inhibits growth of S. aureus.

11. An isolated or purified polypeptide comprising an amino acid sequence having at least 50% sequence identity with the amino acid sequence set forth in SEQ ID NO: 7 or SEQ ID NO: 8, wherein said polypeptide binds the Staphylococcus aureus primary sigma factor polypeptide set forth in SEQ ID NO: 2, and/or wherein said polypeptide inhibits growth of S. aureus.

12. The polypeptide of claim 11, wherein said sequence identity is at least 75%.

13. The polypeptide of claim 11, wherein said sequence identity is at least 95%.

14. An isolated or purified polypeptide comprising amino acids 1-198 of SEQ ID NO: 7 or amino acids 1-149 of SEQ ID NO: 8.

15. A screening method comprising:
(a) contacting a Staphylococcus aureus primary sigma factor polypeptide comprising a bacteriophage binding domain with a test compound in the presence of a bacteriophage polypeptide that specifically binds to said bacteriophage binding domain; and
(b) determining whether the test compound inhibits binding of said bacteriophage polypeptide to said bacteriophage binding domain.

16. The screening method of claim 15, wherein said bacteriophage binding domain comprises an amino acid sequence selected from the group consisting of:
(i) the amino acid sequence set forth in SEQ ID NO: 3;
(ii) the amino acid sequence set forth in SEQ ID NO: 4;
(iii) the amino acid sequence set forth in SEQ ID NO: 5;
(iv) a fragment of (i) or (ii) containing said bacteriophage polypeptide binding domain; and
(v) a variant having at least 95% sequence identity with one of (i), (ii), (iii), and (iv) and containing a domain that is bound by said bacteriophage polypeptide.

17. The screening method of claim 15, wherein said bacteriophage polypeptide is a polypeptide of bacteriophage G1.

18. The screening method of claim 17, wherein said bacteriophage polypeptide comprises amino acids 1-198 of SEQ ID NO: 7 or amino acids 1-149 of SEQ ID NO: 8.

19. The screening method of claim 15, further comprising measuring ability of the test compound to inhibit DNA binding by said Staphylococcus aureus primary sigma factor polypeptide.

20. The screening method of claim 15, further comprising measuring ability of the test compound to inhibit binding between: (i) said Staphylococcus aureus primary sigma factor polypeptide; and (ii) S. aureus Core-RNA polymerase.

21. The screening method of claims 15, further comprising measuring ability of the test compound to inhibit S. aureus RNA polymerase activity.

22. The screening method of claim 15, further comprising measuring bactericidal or bacteriostatic activity of the test compound.

23. The screening method of claim 15, wherein said determining is carried out using a technique selected from the group consisting of Fluorescence Resonance Energy Transfer (FRET), fluorescence polarization, surface plasmon resonance, scintillation proximity assay, biosensor assay, isotermal titration microcalorimetry and phage display.

24. The screening method of claim 15, wherein said test compound is selected from the group consisting of a small molecule, a peptidomimetic compound, a peptide and a polypeptide.

25. A screening method comprising:
(a) contacting (i) a first polypeptide binding domain, (ii) a second polypeptide binding domain and (iii) at least one test compound, wherein said first and second polypeptide binding domains bind specifically with each other,
wherein said first polypeptide binding domain comprises an amino acid sequence selected from the group consisting of: the amino acid sequence set forth in SEQ ID NO: 3; the amino acid sequence set forth in SEQ ID NO: 4; and the amino acid sequence set forth in SEQ ID NO: 5;
wherein said second polypeptide binding domain comprises the amino acid sequence set forth in SEQ ID NO: 7 or SEQ ID NO: 8; and
(b) determining whether said at least one test compound inhibits binding between said first and second polypeptide binding domains.

26. An isolated or purified polynucleotide comprising at least 25 contiguous nucleotides of the nucleic acid sequence set forth in SEQ ID NO: 6, wherein said polynucleotide encodes a polypeptide that binds the Staphylococcus aureus primary sigma factor polypeptide set forth in SEQ. ID NO: 2, and/or wherein said polynucleotide encodes a polypeptide that inhibits growth of S. aureus.

27. An isolated or purified nucleic acid molecule having at least 50% sequence identity with the nucleic acid sequence set forth in SEQ ID NO: 6, wherein said nucleic acid molecule encodes a polypeptide that binds the Staphylococcus aureus primary sigma factor polypeptide set forth in SEQ. ID NO: 2, and/or wherein said nucleic acid molecule encodes a polypeptide that inhibits growth of S. aureus.

28. The nucleic acid molecule of claim 27, wherein said sequence identity is at least 75%.

29. The nucleic acid molecule of claim 27, wherein said sequence identity is at least 95%.

30. An isolated or purified polynucleotide comprising nucleotides 1-597 of SEQ ID NO: 6.

31. An antibacterial compound which inhibits S. aureus primary sigma factor-dependent RNA polymerase activity, wherein said compound binds a bacteriophage binding domain of S. aureus primary sigma factor, and wherein said bacteriophage binding domain comprises an amino acid sequence selected from the group consisting of: the amino acid sequence set forth in SEQ ID NO: 3; the amino acid sequence set forth in SEQ ID NO: 4; and the amino acid sequence set forth in SEQ ID NO: 5.

32. An antibacterial compound which inhibits DNA binding activity of a S. aureus primary sigma factor, wherein said compound binds a bacteriophage binding domain of S. aureus primary sigma factor, and wherein said bacteriophage binding domain comprises an amino acid sequence selected from the group consisting of: the amino acid sequence set forth in SEQ ID NO: 3; the amino acid sequence set forth in SEQ ID NO: 4; and the amino acid sequence set forth in SEQ ID NO: 5.

33. The antibacterial compound of claim 32, wherein said compound mimics the inhibitory activity, and/or the bactericidal or bacteriostatic effect, of the bacteriophage polypeptide comprising amino acids 1-198 of SEQ ID NO: 7 or amino acids 50-198 of SEQ ID NO: 8.

34. An antibacterial compound that has a bactericidal or bacteriostatic effect on Staphylococcus aureus bacteria, wherein said compound binds a bacteriophage binding domain of S. aureus primary sigma factor, and wherein said bacteriophage binding domain comprises an amino acid sequence selected from the group consisting of: the amino acid sequence set forth in SEQ ID NO: 3; the amino acid sequence set forth in SEQ ID NO: 4; and the amino acid sequence set forth in SEQ ID NO: 5.

35. The antibacterial compound of claim 34, wherein said compound mimics the inhibitory activity, and/or the bactericidal or bacteriostatic effect, of the bacteriophage polypeptide comprising amino acids 1-198 of SEQ ID NO: 7 or amino acids 50-198 of SEQ ID NO: 8.


SEQUENCE LISTING (41 sequences)

SEQ ID NO: 1 - DNA, 1107 bp, Staphylococcus aureus
  CDS (1)..(1107)
atgtctgataacacagttaaaattaaaaaacaaacaattgatccgacattaacattagaagatgttaagaagcaattaattgaaaaaggtaaaaaagagggtcatttaagtcatgaagaaattgctgaaaaacttcagaattttgatatcgactctgatcaaatggatgatttctttgatcaattaaatgataatgatatttcactagttaatgaaaaagatagttcagatactgacgagaaactgaatccaagtgatcttagtgcccctccaggtgttaaaataaatgacccagttcgtatgtaccttaaagaaattgggcgtgttaacttattaagtgcacaagaagaaatcgaattagccaaacgtattgaacaaggtgatgaagtagcaaaatcaagacttgcagaagcgaacttacgtttagttgtaagtattgctaaaagatacgtaggtcgtggtatgttattccttgatttaatccaagaaggtaatatgggtcttattaaagctgttgaaaaatttgactttaacaaaggatttaagttttcaacatatgcaacatggtggattagacaagcaatcactcgtgcaattgctgaccaagcacgtacgattcgtatccctgtgcatatggtagaaacaattaataaattaattcgtgttcaacgtcaattattacaggacttaggtcgagatccagcaccagaagaaattggtgaagaaatggatttaccagcagaaaaagttcgtgaaattttaaaaattgcgcaagaacctgtttcattagaaacaccaattggtgaagaagatgatagtcatttaggagactttattgaggatcaggaagcacaaagtccttcagatcatgctgcttatgaattattaaaagagcaattagaagatgtgcttgatacattaactgatagagaagaaaatgtattacgattaagatttggtcttgatgacggcagaacaagaacacttgaagaagttggtaaagttttcggtgttacacgtgaacgtattcgacaaattgaagcaaaagcacttagaaaattaagacatccaagtcgtagtaaacgtttgaaagactttatggattaa

SEQ ID NO: 2 - protein, 368 aa, Staphylococcus aureus
MSDNTVKIKKQTIDPTLTLEDVKKQLIEKGKKEGHLSHEEIAEKLQNFDIDSDQMDDFFDQLNDNDISLVNEKDSSDTDEKLNPSDLSAPPGVKINDPVRMYLKEIGRVNLLSAQEEIELAKRIEQGDEVAKSRLAEANLRLVVSIAKRYVGRGMLFLDLIQEGNMGLIKAVEKFDFNKGFKFSTYATWWIRQAITRAIADQARTIRIPVHMVETINKLIRVQRQLLQDLGRDPAPEEIGEEMDLPAEKVREILKIAQEPVSLETPIGEEDDSHLGDFIEDQEAQSPSDHAAYELLKEQLEDVLDTLTDREENVLRLRFGLDDGRTRTLEEVGKVFGVTRERIRQIEAKALRKLRHPSRSKRLKDFMD

SEQ ID NO: 3 - protein, 242 aa, Staphylococcus aureus
GDEVAKSRLAEANLRLVVSIAKRYVGRGMLFLDLIQEGNMGLIKAVEKFDFNKGFKFSTYATWWIRQAITRAIADQARTIRIPVHMVETINKLIRVQRQLLQDLGRDPAPEEIGEEMDLPAEKVREILKIAQEPVSLETPIGEEDDSHLGDFIEDQEAQSPSDHAAYELLKEQLEDVLDTLTDREENVLRLRFGLDDGRTRTLEEVGKVFGVTRERIRQIEAKALRKLRHPSRSKRLKDFMD

SEQ ID NO: 4 - protein, 75 aa, Staphylococcus aureus
ELLKEQLEDVLDTLTDREENVLRLRFGLDDGRTRTLEEVGKVFGVTRERIRQIEAKALRKLRHPSRSKRLKDFMD

SEQ ID NO: 5 - protein, 67 aa, Staphylococcus aureus
ELLKEQLEDVLDTLTDREENVLRLRFGLDDGRTRTLEEVGKVFGVTRERIRQIEAKALRKLRHPSRS

SEQ ID NO: 6 - DNA, 597 bp, Bacteriophage G1
  CDS (1)..(597)
atgaaattaaagattttagataaagataatgcaacacttaatgtgtttcatcgtaataaggagcacaaaacaatagataatgtaccaactgctaacttagttgattggtaccctctaagtaatgcttatgagtacaagttaagtagaaacggggaatacttagaattaaaaagattacgttctactttaccttcatcttatggtttagatgataataaccaagatattattagagataataaccatagatgtaaaataggttattggtacaaccctgcagtacgcaaagataatttaaagattatagagaaagctaaacaatatggattacctattataacagaagaatatgatgctaatactgtagagcaaggatttagagatattggagttatattccaaagtcttaaaactattgttgttactagatacctagaaggtaaaacagaagaagaattaagaatatttaacatgaaatcagaagagtcacaactgaatgaagcacttaaagagagtgatttttctgtagatttaacttatagtgacttaggacaaatttataatatgttgttattaatgaaaaaaattagtaaatag

SEQ ID NO: 7 - protein, 198 aa, Bacteriophage G1
MKLKILDKDNATLNVFHRNKEHKTIDNVPTANLVDWYPLSNAYEYKLSRNGEYLELKRLRSTLPSSYGLDDNNQDIIRDNNHRCKIGYWYNPAVRKDNLKIIEKAKQYGLPIITEEYDANTVEQGFRDIGVIFQSLKTIVVTRYLEGKTEEELRIFNMKSEESQLNEALKESDFSVDLTYSDLGQIYNMLLLMKKISK

SEQ ID NO: 8 - protein, 149 aa, Bacteriophage G1
NGEYLELKRLRSTLPSSYGLDDNNQDIIRDNNHRCKIGYWYNPAVRKDNLKIIEKAKQYGLPIITEEYDANTVEQGFRDIGVIFQSLKTIVVTRYLEGKTEEELRIFNMKSEESQLNEALKESDFSVDLTYSDLGQIYNMLLLMKKISK

SEQ ID NO: 9 - DNA, 588 bp, Bacteriophage Twort
  CDS (1)..(588)
atgaagttaaaaattaaaaataaatttatgggagtgttagaggttactaattctatgggtgtaactaagttagacgtacccttaagtaacatacatgaatggtatcctttttctaacgcttattcttacaagtataatgtaaaaacaaaagatttagtattaaaacgactacgttcatcactaccagtatcttatgggattgaacgagcgtctaaagagtatgacaaagataaagtatgtaacacagtaacatggataaaccattcagtaaaagacagtaatttacacattattaataaagctaaatcatatgggttacctgttattacagaaaagtatacatatgaagatgtggattatgggtttgcacagttaaatgttatcttttctgaattaaaatctttgattattaatcgttatttagaggataaagatggtagttttattgttaagtttaaaagacacaacccagaaacccaatatcatttagcggtacaagatgctgatgaggttattaataatacctatgatgagctaggtcaaatgtataaaatgttattactaatgaagaaattaagtaagtattaa

SEQ ID NO: 10 - protein, 195 aa, Bacteriophage Twort
MKLKIKNKFMGVLEVTNSMGVTKLDVPLSNIHEWYPFSNAYSYKYNVKTKDLVLKRLRSSLPVSYGIERASKEYDKDKVCNTVTWINHSVKDSNLHIINKAKSYGLPVITEKYTYEDVDYGFAQLNVIFSELKSLIINRYLEDKDGSFIVKFKRHNPETQYHLAVQDADEVINNTYDELGQMYKMLLLMKKLSKY

SEQ ID NO: 11 - DNA, 585 bp, artificial sequence
  Sequence is completely synthesized
atgaanttaaanattnnanataaantnatgnnannnttannggttncnnnntnnnnnggngnaacnannnnnnangtaccnnnnnntaacntanntgantggtancctntnnntaangcttatnnntacaagtnnantnnaaannnnnaananttagnattaaaangantacgttcnncnntaccnnnatcttatggnntngannnnnnnnnnnaagannntnnnanagataannnatgtaananagnnnnntggnnnaaccntncagtannnnannntaatttananattatnnanaaagctaaannatatggnttacctnttatnacagaanantatnnnnntnannntgtngannanggntttnnananntnnnngttatnttnnnnnnnntnaaanctntnnttnttantngntanntagangntaaannngnnnnnnnnntnnnnanntttaanannnannnnnnaganncncaannnnatnnagcnntnnaagannntgatnnnnntntnnatnnnacntatnntganntaggncaaatntataanatgttnttantaatgaanaaantnagtaantan

SEQ ID NO: 12 - protein, 194 aa, Artificial sequence
  Sequence is completely synthesized
MKLKIXXKXXXXLXVXNXXXXTXXXVPXXNXXXWYPXSNAYXYKXXXXXXXLXLKRLRSXLPXSYGXXXXXXXXDXXXXCXXXXWXNXXVXXXNLXIIXKAKXYGLPXITEXYXXXXVXXGFXXXXVIFXXLKXXXXXRYLEXKXXXXXXXFXXXXXEXQXXXAXXXXDXXXXXTYXXLGQXYXMLLLMKKXSK

SEQ ID NO: 13 - DNA, 26 bp, artificial sequence
  Sequence is completely synthesized
cgggatccatgtctgataacacagtt

SEQ ID NO: 14 - DNA, 28 bp, artificial sequence
  Sequence is completely synthesized
cgggatccggtgatgaagtagcaaaatc

SEQ ID NO: 15 - DNA, 28 bp, artificial sequence
  Sequence is completely synthesized
cgggatccgaattattaaaagagcaatt

SEQ ID NO: 16 - DNA, 26 bp, artificial sequence
  Sequence is completely synthesized
cgggatccttaagatttggtcttgat

SEQ ID NO: 17 - DNA, 28 bp, artificial sequence
  Sequence is completely synthesized
acgcgtcgacttatccataaagtctttc

SEQ ID NO: 18 - DNA, 31 bp, artificial sequence
  Sequence is completely synthesized
acgcgtcgacttatgttcttgttctgccgtc

SEQ ID NO: 19 - DNA, 31 bp, artificial sequence; identical to SEQ ID NO: 18
  Sequence is completely synthesized

SEQ ID NO: 20 - DNA, 31 bp, artificial sequence
  Sequence is completely synthesized
acgcgtcgacttaaccttgttcaatacgttt

SEQ ID NO: 21 - DNA, 28 bp, artificial sequence
  Sequence is completely synthesized
cgggatccatgaaattaaagattttaga

SEQ ID NO: 22 - DNA, 28 bp, artificial sequence
  Sequence is completely synthesized
cgggatccaacggggaatacttagaatt

SEQ ID NO: 23 - DNA, 28 bp, artificial sequence
  Sequence is completely synthesized
cgggatccaagattatagagaaagctaa

SEQ ID NO: 24 - DNA, 28 bp, artificial sequence
  Sequence is completely synthesized
cgggatccgaagaagaattaagaatatt

SEQ ID NO: 25 - DNA, 30 bp, artificial sequence
  Sequence is completely synthesized
acgcgtcgacctatttactaatttttttca

SEQ ID NO: 26 - DNA, 31 bp, artificial sequence
  Sequence is completely synthesized
acgcgtcgacttattctgttttaccttctag

SEQ ID NO: 27 - DNA, 31 bp, artificial sequence
  Sequence is completely synthesized
acgcgtcgacttactttaaattatctttgcg

SEQ ID NO: 28 - DNA, 31 bp, artificial sequence
  Sequence is completely synthesized
acgcgtcgacttagtttctacttaacttgta

SEQ ID NO: 29 - DNA, 29 bp, artificial sequence
  Sequence is completely synthesized
ccgctcgaggccatcaggcatggatccgg

SEQ ID NO: 30 - DNA, 45 bp, artificial sequence
  Sequence is completely synthesized
gcgaggctagttacccttaagcttatctttaaatatgaacattcg

SEQ ID NO: 31 - DNA, 27 bp, artificial sequence
  Sequence is completely synthesized
gataagcttaagggtaactagcctcgc

SEQ ID NO: 32 - DNA, 30 bp, artificial sequence
  Sequence is completely synthesized
gaattcgatatcaagcttaattgttatccg

SEQ ID NO: 33 - DNA, 49 bp, artificial sequence
  Sequence is completely synthesized
cggataacaattaagcttgatatcgaattcatcgggaggccgtttcatg

SEQ ID NO: 34 - DNA, 28 bp, artificial sequence
  Sequence is completely synthesized
aactgcagcttattcatgtcttggtatc

SEQ ID NO: 35 - DNA, 32 bp, artificial sequence
  Sequence is completely synthesized
cgggatccatgatagaaatcgaaaaacctaga

SEQ ID NO: 36 - DNA, 34 bp, artificial sequence
  Sequence is completely synthesized
acgcgtcgacactatcttcttttcttaatcctaa

SEQ ID NO: 37 - DNA, 30 bp, artificial sequence
  Sequence is completely synthesized
atgatattgacttattgaataaaattgggt

SEQ ID NO: 38 - DNA, 29 bp, artificial sequence
  Sequence is completely synthesized
accaattttattcaataagtcaatatcat

SEQ ID NO: 39 - DNA, 27 bp, artificial sequence
  Sequence is completely synthesized
atgttgacttaaagaataaaattgggt

SEQ ID NO: 40 - DNA, 48 bp, artificial sequence
  Sequence is completely synthesized
gatagagttgacatccctatcagtgatagagatactgagaacatcagc

SEQ ID NO: 41 - DNA, 49 bp, artificial sequence
  Sequence is completely synthesized
gctgatgtgctcagtatctctatcactgatagggatgtcaatctctatc